(19) United States
(12) Patent Application Publication
(10) Pub. No.: US 2002/0147232 A1
(43) Pub. Date: Oct. 10, 2002

(54) Pharmaceutical compositions comprising desglymidodrine as an active drug substance

(76) Inventors: Claus Sundgreen, Frederiksberg (DK); Ann Christina Schultz, Roskilde (DK); Jimmy Hirschsprung Schlyter, Greve (DK); Peder Mohr Olsen, Kirke Hyllinge (DK)

Correspondence Address: Dike, Bronstein, Roberts & Cushman, Intellectual Property Patent Practice, EDWARDS & ANGELL, LLP, 130 Water Street, Boston, MA, 02109, US

(21) Appl. No.: 09/864,857

(22) Filed: May 23, 2001

(30) Foreign Application Priority Data
May 26, 2000 (DE) PA 2000 00841
Mar. 29, 2001 (DK) PCT/DK01/00214

(51) Int. Cl. A61K 31/375; A61K 31/19; A61K 31/137

(52) U.S. Cl. 514/474; 514/557; 514/649

(57) ABSTRACT

Novel pharmaceutical compositions comprising desglymidodrine or a pharmaceutically acceptable salt thereof as an active drug substance. Desglymidodrine is the active metabolite of the prodrug midodrine. The pharmaceutical composition may be presented in a suitable dosage form for oral, parenteral, mucosal, nasal, sublingual, buccal, topical, vaginal, rectalor ocular etc. administration. A pharmaceutical composition of the invention may be in the form of an immediate and/or modified release composition or it may be designed to release the active drug substance, desglymidodrine, in a relatively fast manner in order to enable a relatively fast onset of the therapeutic effect. The compositions have a suitable shelf-life, i.e. the desglymidodrine contained in the composition is not subject to a significant degradation under storage conditions normally acceptable for pharmaceuticals.

Also disclosed is a method for treating animals such as, e.g. mammals and humans with a novel pharmaceutical composition comprising desglymidodrine.

Furthermore, is disclosed a novel use of desglymidodrine in the treatment of septic shock and to a method for treating mammals (e.g. humans) suffering from septic shock with a sufficient amount of desglymidodrine

This application claims the benefit of U.S. nonprovisional application Ser. No. 09/823,093, filed Mar. 29, 2001.

The present invention relates to novel pharmaceutical compositions comprising desglymidodrine or a pharmaceutically acceptable salt thereof as an active drug substance. Desglymidodrine is the active metabolite of the prodrug midodrine. The pharmaceutical composition may be presented in a suitable dosage form for oral, parenteral, mucosal. nasal, sublingual, buccal, topical, vaginal, rectalor ocular etc. administration. A pharmaceutical composition of the invention may be in the form of an immediate and/or modified release composition or it may be designed to release the active drug substance, desglymidodrine, in a relatively fast manner in order to enable a relatively fast onset of the therapeutic effect.

A pharmaceutical composition according to the invention has a suitable shelf-life, i.e. the desglymidodrine contained in the composition is not subject to a significant degradation under storage conditions normally acceptable for pharmaceuticals.

The invention also relates to a method for treating animals such as, e.g. mammals and humans with a novel pharmaceutical composition comprising desglymidodrine.

Furthermore, the invention relates to a novel use of desglymidodrine in the treatment of septic shock and to a method for treating mammals (e.g. humans) suffering from septic shock with a sufficient amount of desglymidodrine.

Desglymidodrine is the active metabolite of midodrine, i.e. upon administration of midodrine to a patient, midodrine is (enzymatically) metabolised to desglymidodrine. The indications of desglymidodrine are therefore similar to the Indications of midodrine, but as will be explained below desglymidodrine may also be used for novel indications.

The indications of desglymidodrine include symptomatic orthostatic hypotension, syncope, orthostatic intolerance, symptomatic hypotension (e.g. hypotension associated with infections, the convalescent period, surgical operations, delivery, changes in the weather as well as what is called “difficulties in getting started in the mornings”), as well as in the control of hypotensive side effects of hypnotics and psychotropics. Furthermore, desglymidodrine is expected to be effective in the treatment of urinary incontinence. Many of these indications call for a very individual treatment regimen where a basic “all day” treatment supplied with one or more fast onset formulations are very beneficial.

In another aspect, the invention relates to a method for treating hypotension and/or urinary incontinence, the method comprising administration to a patient in need thereof of an effective amount of desglymidodrine in a pharmaceutical composition according to the invention. An interesting pharmaceutical composition according to the invention for this use is a controlled release composition optionally together with one or more fast onset compositions comprising an effective amount of desglymidodrine.

### BACKGROUND OF THE INVENTION

Desglymidodrine is the active form of the prodrug midodrine. Desglymidodrine is a potent adrenergic agonist at the alpha-1 receptors that are mainly localised in resistance and capacitance vessels and in the bladder neck. The effect when given to patients suffering from orthostatic hypotension is an increase in blood pressure and when given to patients suffering from stress incontinence and improvement of continence. Clinical trials of the latter effect have up till now yielded conflicting results.

During development of the compound in the sixties and seventies focus was put on midodrine probably in order to secure a relative long lasting effect when administered orally. New formulation techniques and the search for new indications have turned the inventor's focus to desglymidodrine again.

### DISCLOSURE OF THE INVENTION

As the active metabolite of midodrine, desglymidodrine is a potent drug substance. However, in the past decades focus has been directed to the development of suitable midodrine compositions most likely due to the detailed knowledge of the behaviour of midodrine in clinical studies. However, the need for optimising the treatment of conditions responsive to midodrine has turned the inventors focus on desglymidodrine. By administering pharmaceutical compositions comprising desglymidodrine, the conversion step in vivo from the inactive form, midodrine, to the active form, desglymidodrine, is omitted and, thus, it is anticipated that treatment with desglymidodrine can lead to a fast onset of the therapeutic effect. Furthermore, individual variations with respect to the enzymatic conversion of midodrine to desglymidodrine are of no importance when the treatment is performed with desglymidodrine. Thus, there is a need for the development of suitable pharmaceutical compositions of desglymidodrine for use in the treatment of conditions for which midodrine is indicated. Such compositions provide attractive alternatives or supplements to the treatment of diseases or conditions with a drug substance that acts by stimulating α₁ receptors.

Midodrine is a prodrug, which is activated within the human body by an enzymatic hydrolysis to release the therapeutically active metabolite desglymidodrine. Only very little information is available from the literature concerning desglymidodrine.

The following Is a review of the pharmacokinetics of desglymidodrine as parent compound.

Clinical Studies

The pharmacokinetics of midodrine and desglymidodrine after administration of midodrine have been described by Grobecker (Grobecker H F, Kees F. Pharmacokinetic parameters and haemodynamic actions of midodrine in young volunteers. Int Angiol 1993; 12(2):119-124).

In a Japanese study (Tsutsui S, et at. Pharmacokinetics of midodrine—Bioavailability of midodrine. The clinical report 1987, 21, No.4:1759-1807) the pharmacokinetics of midodrine and desglymidodrine given as prodrug and as parent compound, respectively, was investigated in five healthy volunteers. It was found that the bioavailabily of desglymidodrine was slightly lower when given as parent compound as compared to when given as prodrug. Tmax (time to maximum plasma concentration) for midodrine, desglymidodrine as prodrug and as parent compound, respectively, are given in Table 1 with the results of Grobecker as comparison:

TABLE 1

Tmax (h.)

| study | administered drug | midodrine | desglymidodrine |
|---|---|---|---|
| Tsutsui | midodrine | 1.4 ± 0.2 | 2.0 ± 0.0 |
| Tsutsui | desglymidodrine | | 1.2 ± 0.2 |
| Grobecker | midodrine | 0.5 ± 0.2 | 1.1 ± 0.5 |

Pharmacokinetic data (Tmax) of midodrine and desglymidodrine in two studies Tsutsui (n = 5) and Grobecker (n = 12).

In a pharmacodynamic study (Pittner H. Double-blind trial to compare the effect of midodrine and ST 1059 after oral aministration to volunteers. internal report: DFP 1979) of midodrine, desglymidodrine as parent compound and placebo in seven healthy volunteers, the action of desglymidodrine measured as time to occurrence of pilomotor reaction was shorter, 34±25 min for the parent compound as compared to the prodrug 74±29 min. There was a tendency to a surprisingly longer lasting effect of the parent compound, 160 (135-185) min versus 150 (30-240) min for midodrine.

Engel (Engel K, Havelec L, Klausgraber F, Pramer I. Comparative studies and evaluation of the therapeutic usability of midodrine (Gutron) in the hypotension syndrome. Wiener medizinische Wochenschrift 1974; 124:501-507) has summarised different studies. One of them is a study where it was shown that intravenous injection of 5 mg of desglymidodrine is as potent as 20-25 mg midodrine intravenously with respect to increase of blood pressure. However, the effect of desglymidodrine lasted only 3-4 min.

Only very preliminary conclusions, if any, can be drawn from the above-mentioned studies regarding desglymidodrine. The bioavailability is lower when it is given as parent compound probably due to intraluminal degradation. The time to effect is shorter and the effect is lasting shorter with respect to blood pressure effect, but no difference with respect to pilomotor reaction has been observed. Further studies are needed to characterise the pharmacokinetic of desglymidodrine given as parent compound.

Clinical Indications

The short period of the effect makes desglymidodrine attractive as a quick acting medication in patients with autonomic dysregulation including orthostatic hypotension. A combination with desglymidodrine (or midodrine) in a controlled release formulation would yield an opportunity to create a medication with fast and long lasting effect.

Desglymidodrine acts by a stimulation of α₁ receptors. The prodrug of desglymidodrine, midodrine is used in the treatment of symptomatic orthostatic hypotension. Disorders causing orthostatic hypotension are e.g.,

Generalized primary autonomic failure

ζ Pure autonomic failure or idiopathic orthostatic hypotension (Bradbury-Eggleston syndrome)

ζ Pure autonomic failure with multiple-system atrophy or Shy-Drager syndrome

ζ Acute pandysautonomia (panautonomic neuropathy)

ζ Familial dysautonomia (Riley-Day syndrome)

Partial primary autonomic failure

ζ Dopamine β-hydroxyase deficiency

ζ Postural orthostatic tachycardia syndrome (length-dependent autonomic neuropathy)

ζ Monoamine oxidase deficiency

ζ Pure vasomotor failure

Disorders of idiopathic orthostatic intolerance

ζ Postural orthostatic tachycardia syndrome

ζ Mitral valve prolapse

ζ Due to prolonged bed rest or space flight

ζ Due to asthenic habitus

Disorders of the central nervous system

ζ Tumors (hypothalamic, parasellar, posterior fossa)

ζ Multiple cerebral infarcts

ζ Wernicke's encephalopathy

ζ Tabes dorsalis

ζ Traumatic and inflammatory myelopathies

ζ Parkinson's disease

ζ Hereditary system degenerations

ζ Syringomyelia

ζ Dysautonomia of advanced age

ζ Multiple sclerosis

Systemic diseases with autonomic neuropathy

ζ Botulism

ζ Diabetic neuropathy

ζ Primary systemic amyloidosis

ζ Guillain-Barré syndrome

ζ Porphyria

ζ Lambert-Eaton myasthenic syndrome

ζ Paraneoplastic autonomic neuropathy

ζ Uremic neuropathy

ζ Connective tissue disease

ζ Tangier and Fabry's diseases

ζ Vincristine and heavy metal neuropathies

ζ Leprosy

ζ B₁₂ deficiency

ζ Chronic Chagas' disease

ζ Propafenone neuropathy

Endorcine-metabolic disorders

ζ Primary and secondary adrenocortical insufficiency

ζ Pheochromocytoma

ζ Marked potassium depletion

ζ Severe hypoaldosteronism

Iatrogenic causes

ζ Antihypertensive drugs (α-methyidopa, guanethidine, prazosin, β-blockers)

ζ Psychotropic drugs (phenothiazines, butyrophenones)

ζ Antiparkinsonian drugs (Sinemet, Parlodel)

ζ Vasodilator drugs (nitrates)

ζ Certain illicit drugs (marijuana)

ζ Thoracolumbar sympathectomy

Disorders with diminished cardiac output

ζ Reduced intravascular volume

ζ Acute and chronic blood loss

ζ Fluid loss due to vomiting, diarrhea, diuretics

ζ Gastectomy with the dumping syndrome

ζ Salt-losing nephropathy

ζ Altered capillary permeability

ζ Impaired venous return

ζ Severe varicose veins

ζ Venous obstruction (late pregnancy)

ζ Reflex and pharmacologic vasodilatation

ζ Muscle wasting and prolonged recumbency

ζ Intrinsic cardiac disease

ζ Myocardial infarct

ζ Arrhythmias

ζ Restrictive pericardial/myocardial diseases

Miscellaneous causes

ζ Hyperbradykinnism

ζ Chronic renal hemodialysis

ζ Anorexia nervosa

ζ Reduced aortic compliance

ζ Mastocytosis

ζ Baroreflex failure

Furthermore, midodrine may be used in disorders retrograde ejaculation; disorder of semen ejaculation, or to attenuate symptoms of chronic orthostatic hypotension due to autonomic failure in patients with Bradbury-Eggleston, Shy-Drager syndromes, diabetes mellitus disease and Parkinson's disease.

The prodrug of desglymidodrine, midodrine, is approved in a variety of European and overseas countries including the U.S.A. mainly for the treatment of symptomatic orthostatic hypotension

FDA has recommended a dosing of midodrine of up to 10 mg 3 times daily for the treatment of hypotension, According to FDA, the latest dose must not be given later than 6 pm for safety reasons in order to avoid or reduce the risk of supine hypertension. Other countries recommend that the latest dose must not be given later than 4 hours before bedtime.

Midodrine for use in stress urinary incontinence is a very promising use with a tremendous market potential also due to the ageing population. Current conservative therapeutic approaches are α-sympathomimetics, pelvic floor exercises and estrogens, or surgery, which are rather complementary than competitive.

It is contemplated that desglymidodrine is effective as a drug substance in the treatment of the above-metioned conditions. Furthermore, a completely novel indication for desglymidodrine is described in the following.

Novel Therapeutic Indication

Septic shock is a condition in which bacteremia produces changes in the circulation resulting in critically reduced tissue perfusion. Acute circulatory failure, hypotension and multiorgan failure are characteristic. The pathogenesis is not fully understood but bacterial infection with release of toxins initiate a vicious circle of reactions resulting in vasodilatation and organ hypoperfusion. The cornerstone of the treatment is antibiotics and circulatory support is mandatory to secure organ perfusion. Typically the circulatory disorders are treated with vasopressors initially dopamine followed by addition of norephinephrine and eventually epinephrine.

Norephinephrine has alfa adrenergic and slight beta adrenergic action whereas desglymidodrine only has alpha adrenergic action. Furthermore desglymidodrine does not cross the blood brain barrier in contrast to norepinephrine.

The lack of beta adrenergic action meaning that desglymidodrine has no effect on the heart and the lack of central nervous system stimulation makes desglymidodrine an attractive substitute for norepinephrine in the treatrnent of septic shock as these actions are unwanted.

Active Drug Substances

A pharmaceutical composition according to the invention contains desglymidodrine or a pharmaceutically acceptable salt thereof. From a clinical point of view it may in some cases be advantageous to include midodrine or a pharmaceutically acceptable salt thereof in the composition in order to take advantage of the different pharmacokinetic of the two different substances. As mentioned in the following, the invention also relates to a kit comprising two different compositions, one of the compositions being a composition as described above.

Although the present invention is focused on the use of desglymidodrine, there are cases—as mentioned above—where the concomitant use of midodrine or a midodrine containing composition is relevant. Therefore, in the following is given a description of desglymidodrine as well as midodrine.

With respect to treatment of orthostatic hypotension and the other conditions mentioned above, desglymidodrine or its prodrug midodrine are drugs of choice.

Desglymidodrine as well as midodrine exist in racemic form and in the form of the two enantiomeric species.

Desglymidodrine is also known as ST 1059, alpha-(aminomethyl)-2,5-dimethoxy-benzenemethanol. It may be present in racemic form, i.e. as (±)-desglymidodrine, (±)-ST1059 or (±)-alpha-(aminomethyl)-2,5dimethoxy-benzenemethanol, or in its enantiomericform as (−)-desglymidodrine, (R)-desglymidodrine, (−)-ST1059, (R)-ST1059, (−)-alpha-(aminomethyl)-2,5dimethoxy-benzenemethanol or (R)-alpha-(aminomethyl)-2,5-dimethoxy-benzenemethanol, or in its other enatiomeric form (+)-desglymidodrine, (S)-desglymidodrine, (+)-ST1059, (S)-ST1059, (+)-alpha-(aminomethyl)-2,5-dimethoxy-benzenemethanol or (S)-alpha-(aminomethyl)-2,5dimethoxy-benzenemethanol.

A composition according to the invention may therefore include desglymidodrine in the racemic form (RS), in the enantiomeric form (R), in the enantiomeric form (S) or in mixtures thereof.

In an embodiment according to the invention, a composition contains the active metabolite desglymidodrine (ST 1059), and desglymidodrine is present in the form of (±)-α-(aminomethyl)-2,5-dimethoxy-benzenemethanol (±ST 1059), (+)-α-(aminomethyl)-2,5-dimethoxy-benzenemothanol (+ST 1059), (−)-α-(aminomethyl)-2,5-dimethoxy-benzenemethanol (−ST 1059) or mixtures thereof.

In another embodiment a composition according to the invention contains desglymidodrine in the racemic form (RS), in the enantiomeric form (R), in the enantiomeric form (S) or in mixtures thereof, or it contains at least 90% w/w such as, e.g., at least 95% w/w, at least 97% w/w, at least 98% w/w, at least 99% w/w of desglymidodnne is present in the therapeutically active enantiomeric form. The therapeutically active enantiomeric form of desglymidodrine is contemplated to be (−)-α-(aminomethyl)-2,5-dimethoxy-benzenemethanol (−ST 1059) or the (R) form of desglymidodrine ((R) ST 1059).

Midodrine is also known as ST 1085, or 2-amino-N-[2-(2,5-dimethoxyphenyl)-2-hydroxyethyl]-acetamide. It may be in present in racemic form, i.e. as (±)-midodrine, (±)-ST 1085, or (±)-2-amino-N-[2-(2,5-dimethoxyphenyl)-2-hydroyethyl]-acetamide, (+)-2-amino-N-[2-(2.5-dimethoxyphenyl)-2-hydroxyethyl]-acetamide, or in its enatiomeric form as (−)-midodrine, (R)-midodrine, (−)-ST 1085, (R)-ST 1085, (−)- 2-amino-N-[2-(2,5-dimethoxyphenyl)-2-hydroxyethyl]-acetamide or (R)-2-amino-N-[2-(2,5-methoxyphenyl)-2-hydroxyethyl]-acetamide, or in its other enantiomeric form (+)-midodrine or (S)-midodrine, (+)-ST 1085, or (S)-ST 1085, (+)-2-amino-N-[2-(2,5-dimethoxyphenyl)-2-hydroxyethyl]-acetamide or (S)-2-amino-N-[2-(2,5-dimethoxyphenyl)-2-hydroxyethyl]-acetamide.

In an embodiment, the invention relates to a pharmaceutical kit comprising two different pharmaceutical compositions and one of the compositions is a controlled release composition. In such a kit, one of the compositions may contain midodrine as the active substance and the other may contain desglymidodrine.

In a composition according to the invention desglymidodrine (and whenever relevant midodrine) is present in the form of a pharmaceutically acceptable salt such as a salt formed between desglymidodrine (and whenever relevant midodrine) and an inorganic acid such as e.g., a hydrochloride, a hydrobromide, a hydroiodide, a nitrate, a nitrite, a H₃PO₃ salt, a H₃PO₄ salt, a H₂SO₃ salt, a sulfate, a H₂SO₅ salt, or a salt formed between desglymidodrine (and whenever relevant midodrine) and an organic acid such as organic acids like e.g. H₂CO₃, acetic acid, C₂H₅COOH, C₃H₇COOH, C₄H₉COOH, (COOH)₂, CH₂(COOH)₂, C₂H₅(COOH)₂, C₃H₈(COOH)₂, C₄H₉(COOH)₂, C₅H₁₀(COOH)₂, fumaric acid, maleic acid, lactic acid, tartaric acid, citric acid, ascorbic acid, benzoic acid, salicylic acid and phthalic acid.

A composition according to the invention may comprise a further active drug substance, i.e. the composition may be in the form of a so-called combination composition comprising at least two different active drug substances. The further active drug substance may be any active drug substance, which beneficially is used in combination with desglymidodrine. Interesting examples of further active drug substances are midodrine, steroids like e.g. hydrocortisone or fludrocortisone or somatostin analogoues like e.g. octreotide.

Dosage

In general, the dosage of the active drug substance present in a composition according to the invention depends inter alia on the specific drug substance, the age and condition of the patient and of the disease to be treated.

In general, a composition according to the invention is designed for administration 1-6 times daily. The dosage frequency depends on the specific condition to be treated and on the specific composition used. For example, a nasal composition may be administered only once daily e.g. in order to achieve a relatively fast onset of the therapeutic effect or it may be administered more often in order to treat break-through symptoms. The same applies to e.g. a plain tablet composition, a buccal composition, a rectal composition etc.

A controlled release composition according to the present invention aims at a dosage once, twice or three times daily, preferably once or twice daily. In the present context the term “once daily”/“once-a-day” is intended to mean that it is only necessary to administer the pharmaceutical composition once a day in order to obtain a suitable therapeutic and/or prophylactic response, however, any administration may comprise co-administration of more than one dosage unit, such as, e.g. 2-4 dosage units.

In agreement with the above-mentioned definition of “once daily”/“once-a-day”, “twice daily”/“twice-a-day” is supposed to mean that it is only necessary to administer the controlled release composition at the most twice a day in order to obtain a suitable therapeutic and/or prophylactic response in the patient which can form a basis for an individual supply with the relatively fast onset composition.

Irrespective of the above-mentioned definitiones of “once” and “twice” daily, a dosage unit, which is constructed to deliver the active ingredient after only one daily administration is often preferred by the patient. However, due to individual circumstances some patients may need a new dosage after e.g. 7-18 hours such as, e.g. about 7-8 hours or about 12 or about 18 hours if the patient e.g. has abnormal absorption or bowel transit time. If the individual has a relatively fast bowel transit time, some of the active drug substance may be excreted before the full dosage is released.

Controlled release compositions designed for topical (e.g. transdermal delivery systems), ocular (e.g. ocular delivery systems) or parenteral (e.g. parenteral delivery systems like implants) delivery of desglymidodrine generally aim at less frequent dosage. Thus, a dosage frequency corresponding to 1-2 times a week or 1-2 times a month is often considered appropriate for such delivery systems.

With respect to desglymidodrine, the normal daily dose is from 2.5 to 10 mg three or up to four times daily (calculated as desglymidodrine hydrochloride), i.e. a daily dose of from about 7.5 mg to about 40 mg in the treatment of orthostatic hypertension. However, the daily dose in the treatment of urinary incontinence may be different and, accordingly, a composition according to the present invention typically contains from about 2.5 mg to about 50 mg desgiymidodrine such as, e.g. 2.5 mg, 5 mg, 7.5 mg, 10 mg, 12.5 mg, 15 mg, 17.5 mg, 20 mg, 25 mg, 30 mg, 35 mg, 40 mg, 45 mg or 60 mg. The same applies in general in connection with the treatment of septic shock, where it is anticipated that the dosis range of desglymidodrine is 25-250 μg/kg body weight/min when given as a continuous infusion. The treatment of septic shock is adjusted according to individual susceptibility to desglymidodrine with respect to blood pressure.

In cases, where desglymidodrine is employed in another form, e.g. in another salt form than desglymidodrine hydrochloride, the above-mentioned dosage ranges are of course to be recalculated so that the same dosage is employed on a molar basis.

The total daily doses of desglymidodrine will depend on the indication for the treatment and the individually tolerated doses. The composition or the kit of the present invention provides a possibility of a treatment regimen adapted for the specific patient.

The individual fast onset doses of a composition of the invention may be from 0.2 mg to 10 mg, preferreably from 0.5 mg to 7.5 mg such as of 0.75 mg, 1 mg, 1.25 mg, 1.5 mg, 2 mg, 2.5 mg, 3 mg, 4 mg, or 5 mg.

As discussed above, desglymidodrine may be present as the racemic form or in one of its enantiomeric forms, preferably the therapeutically active enantiomeric form. In those cases where desglymidodrine is present in its therapeutically active enantiomeric form a reduction in the above-mentioned dosage ranges may be relevant.

With respect to the dosage in those cases where midodrine also is employed it is envisaged that the same dosages as mentioned above are relevant. Thus when both midodrine and desglymidodrine are employed either in combination or in a kit according to the invention, then the total dose of desglymidodrlne and midodrine is contemplated to be in the above-mentioned dosage ranges on a molecular basis.

Pharmaceutical Compositions

A pharmaceutical composition of the invention comprises desglymidodrine or a pharmaceutically acceptable salt thereof together with one or more pharmaceutically acceptable excipients.

A composition according to the invention may be in the form of a solid, semi-solid or fluid composition. Examples of solid compositions are e.g. tablets such as, e.g, conventional tablets, effervescent tablets, melt tablets, coated tablets (e.g. film coated, enteric coated, controlled release coated), sublingual tablets, buccal tablets, capsules, sachets, powders, granules, pellets, microcapsules, microspheres, nanoparticles etc. Examples of semi-solid compositions are e.g. ointments, creams, liniments, chewing gums, hydrogels, pastes, suppositories, enemas, etc. Examples of fluid and/or liquid compositions are e.g. drops, dispersions, solutions, emulsions, suspensions, liposomes, sprays, mixtures, syrups, gels, hydrogels, aerosols. A composition according to the invention may be suitable for administration via the oral, peroral, buccal, sublingual, rectal, vaginal, nasal, ocular, topical and parenteral route. Examples on suitable compositions are also e.g. nasal compositions such as, e.g. nasal spray or nasal powder compositions, compositions for pulmonary administration such as, e.g. inhalators, chewing compositions such as, e.g., chewing gum, etc. Especially suited compositions for a fast onset are compositions in fluid form and in the form of nasal compositions as well as quick release tablets. Especially suited compositions for controlled or modified release of desglymidodrine are controlled release pellets, controlled release tablets, controlled release granules, controlled release capsules, transdermal delivery systems (e.g. patches, plasters), ocular delivery systems (e.g. lenses), parenteral delivery systems (e,g. implants, oil-based compositions) and bioadhesive delivery systems (e.g. for oral, buccal or topical use). The controlled release of desglymidodrine may be obtained by employment of various controlled release techniques such as, e.g. matrix tablets, application of controlled release coating(s) etc.

In an embodiment of the invention, the composition is in the form of tablets having a disintegration time of at the most about 2.5 min such as, eg at the most about 30 sec, at the most about 45 sec. at the most about 1 min, at the most about 1.5 min or at the most about 2 min.

Generally, a composition of the invention has a shelf-life at room temperature of at least 6 months such as, e.g. at least 1 year, at least 1.5 years, at least 2 years, at least 2, 5 years, 3 years, 4 years or 5 years. Shelf-life is defined as the time period in which a change in the content of desglymidodrine in the composition is at the most ±10% w/w such as, e.g., at the most ±7.5% w/w or ±5% w/w; in the present context the shelf-life is determined under conditions of room temperature, 25° C. and a relative humidity of 60%. The compositions of the invention are thus stable, i.e. no significant degradation of desglymidodrine is observed during normal storage conditions.

Plain Compositions

In the embodiments of the invention in which the composition is not specifically designed to release desglymidodrine in a controlled manner, the release kinetics of desglymidodrine from the composition corresponds to that of a plain release tablet. The release kinetic of desglymidodrine from such compositions corresponding to a zero or a first order release, a mixture of zero and first order release, or any other order of release such as, e.g. 1½, second, third or fourth order release (the same applies also to controlled release compositions of the invention).

The invention also relates to a pharmaceutical composition comprising desglymidodrine (ST 1059) or a pharmaceutically acceptable salt thereof, the composition being adapted to provide desglymidodrine in such a manner that a relatively fast therapeutically effective concentration of deaglymidodrine is obtained after administration of the composition.

Accordingly, a composition of the invention may be adapted to release desglymidodrine in such a manner that a relatively fast therapeutic effective concentration of desglymidodrine is obtained after administration of the composition e.g. to obtain an onset of action at the most 15 min after administration such as, e.g. at the most about 1 min, at the most about 2 min, at the most about 3 min, at the most about 4 min, at the most about 5 min, at the most about 7.5 min, at the most about 10 min or at the most about 12.5 min after administration,

Alternatively, a composition of the invention may be designed in such a manner that a therapeutically effective concentration is obtained within 90 min such as, e.g. within 60 min, within 45 min, within 30 min, within 20 min, within 15 min, within 10 min, within 5 min from administration of the composition.

For such compositions (i.e. not controlled release compositions) a relatively fast peak plasma concentration of desglymidodeine is obtained about 1 min-6 hours such as, e.g. about 5 min-6 hours, about 10 min-5 hours, about 15 min - 5 hours, about 0.5-6 hours, about 1-6 hours, about 2-5.5 hours, or about 2.5-5.2 hours after administration.

Controlled Release Compositions

In a further aspect, the invention relates to a controlled release pharmaceutical composition comprising desglymidodrine. The controlled composition may be designed for administration via the oral, parenteral, topical, buccal, vaginal, ocular etc. route.

A controlled release pharmaceutical composition of desglymidodrine according to the invention may also be designed for administration once, twice or three times daily, preferably once or twice daily, i.e. a therapeutically effective concentration of desglymidodrine is maintained for a period of at least 6 hours such as, e.g. at least 7 hours, at least 8 hours, at least 9 hours or at least about 10-16 hours followed by a wash out period of about 8-12 hours in order to avoid side effect with respect to supine hypertension. Such side effects are well known after administration of midodrine.

A controlled release composition provides a base line plasma concentration, which during most of the day is therapeutically effective. When a higher concentration is needed, only a minor supply of active drug substance is necessary to obtain a very fast relief from symptoms. If the base line plasma concentration is absent, it would be necessary to use a relative higher fast onset dose to reach the high therapeutically effective level. The high therapeutically effective level may be due to individual circumstances in the patient or may be a consequence of physical routines and/or the nature of the underlying disease. The situations and symptoms are often well recognized and experienced by the patient himself. The composition according to the present invention is a superior tool for obtaining an optimal treatment with a minimum of active drug substance.

In the present context a therapeutically effective concentration of desglymidodrine is defined as a plasma concentration of desglymidodrine of at least about 3 ng/ml such as, e.g. at least about 3.2 ng/ml, at least about 3.5 ng/ml, at least about 3.7 ng/ml, at least about 4.0 ng/ml, at least about 4.2 ng/ml, at least about 4.5 ng/ml, at least about 4.7 ng/ml or at least about 5 ng/ml.

The controlled release pharmaceutical composition may be designed to release desglymidodrine in such a manner that a relatively fast peak plasma concentration of desglymidodrine is obtained followed by a prolonged and relatively constant plasma concentration of desglymidodrine. However, the patient may due to individual needs or because of activities during the day experience situations where an increase in the plasma concentration is needed for an optimal treatment regimen. Therefore, the patient may on an individual basis supply the controlled release composition with one or more administrations of a quick release composition or another composition providing a relatively fast onset.

A composition according to the invention may comprise one or more further active drug substances.

Pharmaceutical Kit

In a further aspect, the invention relates to a pharmaceutical kit comprising a plain composition and a controlled release composition.

Accordingly, a pharmaceutical kit of the invention comprises

i) a relatively fast onset pharmaceutical composition, wherein the composition is adapted to provide desglymidodrine in such a manner that a relatively fast therapeutically effective concentration of desglymidodrine is obtained after administration, and

ii) a controlled release pharmaceutical composition, wherein the composition is adapted to release desglymidodrine in such a manner that a therapeutically effective plasma concentration of desglymidodrine is maintained for at least about 2 hours, such as, e.g. at least about 3 hours, at least about 4 hours, at least about 5 hours, at least about 6 hours, at least about 7 hours, at least about 8 hours or at least about 9 hours.

In alternative embodiments, the invention relates to a pharmaceutical kit comprising

i) a relatively fast onset pharmaceutical composition comprising midodrine, wherein the composition is adapted to provide midodrine in such a manner that a relatively fast therapeutically effective concentration of midodrine is obtained after administration, and

ii) a controlled release pharmaceutical composition of desglymidodrine, wherein the composition is adapted to release desglymidodrine in such a manner that a therapeutically effective plasma concentration of desglymidodrine is maintained for at least about 2 hours, such as, e.g. at least about 3 hours, at least about 4 hours, at least about 5 hours, at least about 6 hours, at least about 7 hours, at least about 8 hours or at least about 9 hours.

A pharmaceutical kit comprising

i) a relatively fast onset pharmaceutical composition of desglymidodrine, wherein the composition is adapted to provide desglymidodrine in such a manner that a relatively fast therapeutically effective concentration of desglymidodrine is obtained after administration, and

ii) a controlled release pharmaceutical composition comprising midodrine, wherein the composition is adapted to release midodrine in such a manner that a therapeutically effective plasma concentration of midodrine is maintained for at least about 2 hours, such as, e.g. at least about 3 hours, at least about 4 hours, at least about 5 hours, at least about 6 hours, at least about 7 hours, at least about 8 hours or at least about 9 hours.

International patent application No. PCT/DK01/00213 being in possession of the same assignee as the present application discloses specific controlled release compositions for oral use comprising midodrine andlor desglymidodrine. Therefore, for states in which the present application is co-pending with a national phase of the above-mentioned patent application, such specific compositions may conveniently be outside the scope of the present invention.

The various compostions may be prepared by a person skilled in the art optionally with guidance from Remington's Pharmaceutical Sciences and with guidance from the disclosure and examples of international patent application No. PCT/DK01/00213 and PCT/DK01/00214.

Formulation Techniques

Any relevant formulation technique for preparing pharmaceutical compositions may be applied when formulating a composition according to the invention. A person skilled in the art of pharmaceutical formulation techniques can find guidance in the handbook Remington's Pharmaceutical Sciences and in the non-limiting Examples herein.

In principle any relevant non-controlled or controlled formulation technique for preparing an oral non-controlled or controlled release composition may be applied. Thus, the dosage form may be in the form of a liquid having e.g. particles dispersed in a dispersion medium or it may be in the form of a single or a multiple unit dosage form intended for use as such as for dispersing in a dispersion medium before use.

In the following is given a brief non-limiting description of controlled release formulation techniques. Examples of different controlled release technologies especially for the preparation of compositions for oral use are:

1. Single units

1.1 Coated matrix

1.2 Double or triple compression

1.3 Multilayer coating

1.4 Coated single units (e.g. enteric coating and employment of amylose e.g. as a colon degradable excipient).

2. Multiple units

2.1 Units having a controlled release coating

2.2 Units having a controlled release matrix

2.3 Units having a controlled release compression coating

2.4 Units with a multilayer coating.

Other relevant controlled release compositions are e.g. patches, plasters (for e.g. transdermal use), emulsions, imiants, dispersions (for e.g parenteral, topical, vaginal or ocular use), or incorporation of desglymidodrine into a suitable delivery system (for e.g. vaginal, parenteral, ocular, buccal use).

Multiple Unit Systems

The units comprise pellets, granules, crystals, mini tablets or mixtures thereof. A relatively fast relase can be obtained by use of an uncoated unit. A controlled release can be obtained by the application of a controlled release coating or by formulating the unit as a matrix or a coated matrix. A delayed release can be obtained by the use of an enteric polymer or amylose, or by having units compressed as described in the triple compression technology, cf. the examples herein.

In specific embodiments, a composition according to the invention is in the form of a solid dosage form.such as, e.g., tablets, capsules, sachets, solid dispersion, crystals, granules and the like.

A controlled release composition according to the invention can also comprise at least two parts such as at least a first and a second part, each part contains desglymidodrine, and the first part being adapted to release desglymidodrine in a controlled manner during the first 0-14 such as, e.g. 0-11 hours, 0-10 hours, 0-9 hours, 1-8 hours, 1-7 hours, 1-6 hours, 2-5 hours, 2-4 hours or 2-3 hours after oral intake and the second part being adapted to release desmidodrine starting at least 2 hours such as, e.g. at least 3 hours, at least 4 hours, at least 5 hours or at least 6 hours after oral intake.

In such a composition at least one of the at least two parts is present in the composition in the form of a multiplicity of individual units such as, e.g. pellets or minitablets. The individual pellets or minitablets may be the same or different, i.e. they may have the same or different dissolution characteristics.

A composition according to the invention may also as individual units contain minitablets, i.e. be in the form of a multiple unit dosage form comprising at least two different types of minitablets, the first type of minitablets corresponding to the first part and the second type of minitablets corresponding to the second part. In the present context a minitablet is a tablet having a size in a range corresponding to from about 0.7 mm to about 7 mm in diameter such as, e.g., in a range corresponding to from about 1 to about 7 mm, from about 1.5 to about 6 mm, from about 2 mm to about 5 mm, from about 2 mm to about 4 mm such as in a range corresponding to from about 2 to about 3 mm in diameter.

A controlled release composition according to the invention may also as individual units contain relatively large crystals of the active drug substance. In such cases, the size of the unit is at the most about 1 mm such as, e.g., in a range corresponding to from about 0.1 to about 1 mm, from about 0.2 mm to about 0.8 mm, from about 0.2 mm to about 0.7 mm or from about 0.3 mm to about 0.7 mm.

A composition or a controlled release composition according to the invention may be in the form of a multiple unit dosage form, wherein the first or the second part is in the form of minitablets, in the form of pellets or in the form of large crystals of the active drug substance.

Moreover, at least two fractions may be present in a tablet such as, e.g. a multilayer tablet and the at least first and the second part are each comprised in a layer in the tablet.

Furthermnore, a composition according to the invention may comprise a third part adapted to release desglymidodrine relatively fast from the composition and/or a fourth part adapted to release desglymidodnne from the compositon 6-10 hours after oral intake.

In one embodiment the third and/or, if present, the fourth part comprise pellets or minitablets or are a layer in a tablet.

With respect to release kinetics, a composition according to the invention may have a first part, a second part, a third part and/or a fourth part which have a release kinetic corresponding to a zero or a first order release or a mixture of zero and first order release Other orders of release may be 1.5, 2, 3 or 4.

All the abovementioned combinations of different types of compositions or formulation techniques apply, whenever relevant, mutatis mutandi to a composition of the invention. The same applies to the combination of the controlled release part or composition and the fast release part or composition of a kit of the invention

Pharnaceutically Acceptable Excipients

Apart from the active drug substance in the composition, a pharmaceutical composition according to the invention may further comprise pharmaceutically acceptable excipients.

In the present context, the term “pharmaceutically acceptable excipient” is intended to denote any material, which is inert in the sense that it substantially does not have any therapeutic and/or prophylactic effect per se. A pharmaceutically acceptable excipient may be added to the active drug substance with the purpose of making it possible to obtain a pharmaceutical composition, which has acceptable technical properties.

The choice of pharmaceutically acceptable excipients depends inter alia on the specific composition and the intended administration route.

Water is very often an acceptable excipient and this is also the case in the present context. However, it is contemplated that there may have been described solutions or other kind of compositions containing desglymidodrine and water e.g. in connection with testing of the in vivo behaviour of desglymidodrine. An example is e.g. employment of 3H-desglymidodrine in a study by Kolassa et al. (Archives Internationals de Pharmacodynamie et Thérapie, 1979, vol. 238, 96-104). In this paper it is not explicity stated that e.g. water is employed as solvent, but if necessary and for jurisdictions where it may be relevant, it may be necessary to disclaim compositions containing a specific desglymidodrine and a specific solvent (e.g. the labelled desglymidodrine in the specific concentration used and in the specific solvent used (which in this case may be water).

Fillers/diluents/binders may be incorporated such as sucrose, sorbitol, mannitol, lactose (e.g., spray-dried lactose, α-lactose, β-lactose, Tabletose®, various grades of Pharmatose®, Microtose or Fast-Floc®), microcrystalline cellulose (e.g., various grades of Avicel®, such as Avicel® PH101, Avicel® PH102 or Avice® PH105, Elcema® P100, Emcocel®, Vivacel®, Ming Tai® and Solka-Floc®), hydroxypropylcellulose, L-hydroxypropylcellulose (low-substituted) (e.g L-HPC-CH31, L-HPC-LH11, LH 22, LH 21, LH 20, LH 32, LH 31, LH30), dextrins, maltodextrins (e.g. Lodex® 5 and Lodex® 10), starches or modified starches (including potato starch, maize starch and rice starch), sodium chloride, sodium phosphate, calcium phosphate (e.g. basic calcium phosphate, calcium hydrogen phosphate), calcium sulfate, calcium carbonate, gelatine, polyvinylpyrrolidone (30, 90, Kollidon VA 64), and sodium carboxymethylcellulose.

Disintegrants may be used such as cellulose derivatives, including microcrystalline cellulose, low-substituted hydroxypropyl cellulose (e.g. LH 11, LH 22, LH 21, LH 20, LH 32, LH 31, LH30); starches, including potato starch; croscarmellose sodium (i.e. cross-linked carboxymethylcellulose sodium salt; e.g. Ac-Di-Sol®); alginic acid or alginates, insoluble polyvinylpyrrolidone (e.g. Polyvidon® CL, Polyvidon® CL-M, Kollidon® CL, Polyplasdone® XL, Polyplasdone® XL-10); sodium carboxymethyl starch (e.g. Primogel® and Explotab® ).

Glidants and lubricants may be incorporated such as stearic acid, metallic stearates, talc, waxes and glycerides with high melting temperatures, colloidal silica, sodium stearyl fumarate, polyethylenglycols and alkyl sulphates.

Surfactants may be employed such as non-ionic (e.g., polysorbate 20, polysorbate 21, polysorbate 40, polysorbate 60, polysorbate 61, polysorbate 65, polysorbate 80, polysorbate 81, polysorbate 85, polysorbate 120, sorbitane monoisostearate, sorbitanmonolaurate, sorbitan monopalmitate, sorbitan monostearate, sorbitan monooleate, sorbitan sesquioleate, sorbitan trioleate, glyceryl monooleate and polyvinylalkohol), anionic (e.g., docusate sodium and sodium lauryl sulphate) and cationic (e.g., benzalkonium chloride, benzethonium chloride and cetrimide) or mixtures thereof. Examples of amphoteric surfactants are 1,2-diacyl-L-phosphatidylcholine, N-lauryl-N,N-dimethylglycine, alkylaminopropionic acid, alkyliminodipropionic acid, and dimethyl-(3-palmitamidopropyl)-aminoacetate,

Other appropriate pharmaceutically acceptable excipients may include colorants, organoleptic improving agents, taste-improving agents, flavouring agents, antioxidants, vitamins, pH adjusting agents, solubilizing agents, wetting agents and buffering agents.

For application to the rectal or vaginal mucosa suitable compositions according to the invention include suppositories (emulsion or suspension type), solutions, enemas and rectal gelatin capsules (solutions or suspensions) as well as the compositions mentioned in the Examples herein. Appropriate suppository bases include cocoa butter, esterified fatty acids, glycerinated gelatin, and various water-soluble or dispersible bases like polyethylene glycols and polyoxyethylene sorbitan fatty acid esters. Various additives like e.g. enhancers may be added.

For application to the nasal mucosa, nasal sprays and aerosols for inhalation are suitable compositions according to the invention. In a typically nasal composition, the active drug substance is dissolved or dispersed in a suitable vehicle. The pharmaceutically acceptable vehicles and excipients present in the composition are all selected in accordance with conventional pharmaceutical practice in a manner understood by a person skilled in the art of formulating pharmaceutics (for specific examples, see the examples herein).

For application to the oral cavity or skin, the compositions according to the invention may contain conventionally non-toxic pharmaceutically acceptable carriers and excipients including microspheres and liposomes. The compositions include creams, ointments, lotions, liniments, gels, hydrogels, solutions, suspensions, sticks, sprays, pastes, dressings, bandages, plasters, and the like. The pharmaceutical acceptable carriers or excipients may include emulsifying agents, antioxidants, buffering agents, preservatives, humectants, penetration enhancers, chelating agents, gelforming agents, ointment bases, perfumes and skin protective agents.

Examples of emulsifying agents are naturally occurring gums, e.g. gum acacia or gum tragacanth, naturally occurring phosphatide, e.g soybean lecithin and sorbitan monooleate derivatives.

Examples of enhancers are alcohols (such as, e g., ethanol, lauryl alcohol), esters (e.g. glycerol monolaurate), salicylic acid, anionic surfactant, (e.g. sodium dodecyl sulfate), cationic surfactant (e.g. cetyltrimethyl ammonium bromide), non-ionic surfactants (e.g. polysorbates), phosporlipids (e.g. phosphtidyl choline), urea, propylene glycol, DMSO, triethanolamine, N.Ndimethylacetamide, N,N-dimethylformamide, 2-pyrrolidone and derivatives thereof, tetragydrofuryl alcohol, Azone.

Modified Release Coating

A unit comprised in a composition according to the invention or the composition itself may be coated with a modified release coating.

The modified release coating is a substantially water-insoluble but water-diffusible coating.

The modified release coating may be applied on the multiple units or on the single units from a solution and/or suspension preferably in an aqueous solvent, but an organic coating composition may also be applied. The modified release coating may also be applied as a compression coating comprising a dry mixture of polymer(s) and the e.g. the active drug substance.

Examples of matrix-forming agents are hydroxypropylmethylcellulose such as, e.g., 1828, 2208, 2906 or 2910 according to USP, hydroxypropylcellulose, micronised ethylcellulose, low-substituted hydroxypropylcellulose (LH 20, 21, 31).

Examples of film-forming agents which are suitable for use in accordance with the present invention are agents selected from the group consisting of cellulose derivatives such as, e.g., ethylcellulose, cellulose acetate, cellulose propionate, cellulose butyrate, cellulose valerate, cellulose acetate propionate; acrylic polymers such as, e.g., polymethyl methacrylate; vinyl polymers such as, e.g., polyvinyl acetate, polyvinyl formal, polyvinyl butyryl, vinyl chloride-vinyl acetate copolymer, ethylene-vinyl acetate copolymer, vinyl chloride-propylene-vinyl acetate copolymer; silicon polymers such as, e.g., ladder polymer of sesquiphenyl siloxane, and colloidal silica; polycarbonate; polystyrene; polyester; coumarone-indene polymer; polybutadiene; and other high molecular synthetic polymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In one preferred embodiment, the acrylic coating is an acrylic resin lacquer used in the form of an aqueous dispersion, such as that which is commercially available from Röhm Pharma under the tradename Eudragit®. In further preferred embodiments, the acrylic coating comprises a mixture of two acrylic resin lacquers commercially available from Röhm Pharma under the tradenames Eudragit® RL 30 D and Eudragiy® RS 30 D, respectively. Eudragit® RL 30 D and Eudragit® RS 30 D are copolymers of acrylic and methacrylic esters with a low content of quaternary ammonium groups, the molar ratio of ammonium groups to the remaining neutral (meth)acrylic esters being 1:20 in Eudragit® RL 30 D and 1:40 in Eudragit® RS 30 D. Eudragit® RL/RS mixtures are insoluble in water and in digestive fluids. However, coatings formed from the same are swellable and permeable in aqueous solutions and digestive fluids. The Eudragit® RL/RS dispersions may be mixed together in any desired ratio in order to ultimately obtain a modified release formulation having a desirable dissolution profile. The most desirable modified release formulations may be obtained from a retardant coating based on Eudragit® NE 30D, which is a neutral resin having a molecular weight of 800,000.

Examples of enteric polymers are cellulose acetate phthalate, cellulose acetate trimellitate, hydroxy propyl methyl cellulose acetate phthalate, hydroxy propyl methyl cellulose acetate succinate, carboxy methyl ethyl cellulose, polyvinyl acetate phthalate, copolymer of vinyl acetate and crotonic acid and poly(methacrylic acid, ethacrylate), and Eudragit® S 12.5, Eudragit® S 100, Eudragit® FS 30D (all from Röhm), Sureteric® (from Colorcom), Aquateric® (from FMC) or HPMCP (from Shin-Etsu).

The amount of coating applied is adapted so as to obtain a predetermined dissolution characteristic of the composition.

However, the amount of coating applied should also be adapted so that there will be no rupturing problems.

The coating may be admixed with various excipients such as plasticizers, anti-adhesives such as, e.g., colloidal silicium dioxide, inert fillers, lipophilic agents such as, e.g, stearic acid, capric acid or hydrogenated castor oil, colon targeting excipients such as, e.g. amylose, ethylcellulose, Eudragit S 12.5 etc., and pigments in a manner known per se.

Tackiness of the water-dispersible film-forming substances may be overcome by simply incorporating an anti-adhesive in the coating. The anti-adhesive is preferably a finely divided, substantially insoluble, pharmaceutically acceptable non-wetting powder having anti-adhesive properties in the coating. Examples of anti-adhesives are metallic stearates such as magnesium stearate or calcium stearate, microcrystalline cellulose, or mineral substances such as calcite, substantially water-insoluble calcium phosphates or substantially water-insoluble calcium sulphates, colloidal silica, titanium dioxide, barium sulphates, hydrogenated aluminium silicates, hydrous aluminium potassium silicates and talc. The preferred anti-adhesive is talc. The anti-adhesive or mixture of anti-adhesives is preferably incorporated in the coating in an amount of about 0.1-70% by weight, in particular about 1-60% by weight, and preferably about 8-50% by weight of the film layer. By selecting a small particle size of the talc, a larger surface area is obtained; the consequent higher anti-adhesive effect makes it possible to incorporate smaller amounts of specific anti-adhesives.

The units or the composition may further comprise an outer film layer.

In one aspect, the outer second layer comprises a water-based film-forming agent which prevents adhesion between the units at elevated temperatures and imparts flowability to the units, the water-based film-forming agent being anti-adhesive at temperatures above about 40° C., especially temperatures above about 50° C., such as a temperature between about 60° C. and about 120° C., and being selected from diffusion coating materials such as ethylcellulose or enteric coating materials such as anionic poly(meth)acrylic acid esters, hydroxypropylmethylcellulosephthalate, celluloseacetatephthalate, polyvinylacetatephthalate, polyvinylacetatephthalate-crotonic acid copolymerisates, or mixtures thereof, or water-soluble coating materials such as water-soluble cellulose derivatives, e.g. hydroxypropylcellulose, carboxymethylcellulose, methylcellulose, propylcellulose, hydroxyethylcellulose, carboxyethylcellulose, carboxymethylhydroxyethylcellulose, hydroxymethylcellulose, carboxymethylethylcellulose, methylhydroxypropylcellulose or hydroxypropylmethylcellulose.

Examples of plasticizers for use in accordance with the present invention include triacetin, acetylated monoglyceride, rape oil, olive oil, sesame oil, acetyl tributyl citrate, acetyl triethyl citrate, glycerin, sorbitol, diethyloxalate, diethylmalate, diethylmaleate, diethylfumarate, diethylsuccinate, diethylmalonate, dioctylphthalate, dibutylsebacetate, triethylcitrate, tributylcitrate, glyceroltributyrate, polyethyleneglycol, propyleneglycol, 1,2-propyleneglycol, dibutylsebacate, diethylsebacate and mixtures thereof. The plasticizer is normally incorporated in an amount of less than 20% by weight, calculated on the dry matter content of the coating composition.

A relatively fast onset composition according to the invention may be any composition well known in the art to provide a relative fast release.

With respect to nasal vehicles, polyethyleneglycols is especially preferred such as more n-ethylene glycols represented by the following formula

H(OCH₂CH₂)ₚOH

wherein p is an integer in the range of 1 to 14. Examples of n-ethylene glycols are monoethylene glycol (1EG), di ethylene glycol (2EG), triethylene glycol (3EG), tetraethylene glycol (4EG), penta ethylene glycol (5EG), hexaethylene glycol (6EG), heptaethylene glycol (7EG), octaethylene glycol (8EG), nonaethylene glycol (9EG), decaethylene glycol (10EG), undecaethylene glycol (11EG), dodecaethylene glycol (12EG), tridecaethylene glycol (13EG), and tetradecaethylene glycol (14EG). The ethylene glycols may be used in the form of the single compounds or as a mixture of two or more n-ethylene glycols, e.g. commercial products such as polyethylene glycol 200 (PEG 200), polyethylene glycol 300 (PEG 300) or polyethylene glycol 400 (PEG 400). The polyethyleneglycols may be used in combination with glycofurols (α-[(tetrahydro-2-furany)methyl]-ω-hydroxy-poly(oxy-1,2-ethanediyl)). The latter may also be used separately.

The volume of a nasal dosage is preferably within 500 μl such as within 300 μl such as in a range of 10-250 μl.

Composition Characteristics

Due to the rather short half-life of approximately 3 hours desglymidodrine normally must be administered 24 times daily. Considering the chronic nature of the diseases in question, which requires a long-term treatment as well as the correlation between plasma levels and the incidence and severity of adverse events, the development of a controlled release form is highly desired. As discussed earlier, the development of a non-controlled release form is also important from a clinical point of view and the same applies to the development of a composition intended for a relatively fast onset.

With respect to midodrine, the prodrug of desglymidodrine, it has now been found that absorption takes place through the whole gastrointestinal tract. Thus, it has been found that when midodrine reaches the colon (about 8 hours after intake of a single unit capsule containing midodrine) the prodrug midodrine is not measured in plasma at least not at a therapeutic level while the extent of absorption of the active metabolite is identical to that of a solution. In other words, with respect to absorption from the colon it has been found that it is not midodrine, which is measured after oral intake of midodrine but instead it is the active metabolite desglymidodrine itself.

After colon absorption a maimum plasma concentration of desglymidodrine is found to take place at approximately 3 hours after application, i.e. Tₘₐₓ corresponds to approx. 3 hours. In contrast thereto, a tₘₐₓ of about 1-2 hour for desglymidodrine is observed after oral intake of midodrine and the corresponding value for midodrine itself is a Tₘₐₓ of about 30 min.

The finding that midodrine is converted to the active metabolite before or during absorption from the colon is of importance with respect to the dissolution requirements of a composition of the present invention. A further important issue is the fact that FDA has recommended that the latest dose of midodrine is taken not later than 6 pm for safety reasons, thus a wash out period through the night is desirable.

Based on the above findings and the therapeutic needs, the present inventors have developed a composition containing desglymidodrine. A composition of the invention may be suitable for use as the sole medicament or it may be suitable for use in combination with another drug substance such as, e.g., midodrine. For use in the latter case, the invention provides a kit containing at least two different parts at least one of which contains desglymidodrine.

Basically two different compositions are of specific interest, i.e. a composition for normal or relatively quick release of desglymidodrine and a composition for controlled release of desglymidodrine. Besides those two kind of compositions, the invention relates to a kit comprising two compositions, the one being a normal or relatively quick release composition and the other being a controlled release composition. The compositions of the kit generally contains desglymidodrine, but there may be situations where it is advantategous to have the normal/quick release composition having a content of midodrine or, alternatively, to have the controlled release composition being a midodrine controlled release compositon.

In the present context the term kit is intended to include

i) a package comprising at least a first and a second pharmaceutical composition, wherein the first composition is designed to release the desglymidodrine relatively fast in order to obtain a relatively fast onset of the therapeutic effect and the second composition is in the form of a controlled release composition (cf. a co-pending patent application by the same Applicant) which is designed to give a release pattern as described below in order to utilize the possibility of having the active drug substance absorbed not only in the upper part of the gastrointestinal tract but also during its passage through colon, the first and the second composition may be of the same kind, e.g. in the form of tablets or capsules or they may be in the form of two different types of pharmaceutical compositions e.g. the first composition may be in the form of plain tablets or a nasal spray and the second composition may be in the form of controlled release tablets or capsules, and

ii) a pharmaceutical composition which include a first and a second part, wherein the first part is designed to release desglymidodrine relatively fast in order to obtain a relatively fast onset of the therapeutic effect and the second part is a controlled release part (cf. a co-pending patent application by the same Applicant) which is designed to give a release pattern as described below in order to utlize the possibility of having the active drug substance absorbed not only in the upper part of the gastrointestinal tract but also during its passage through colon, and the first and the second part are presented in the form of a single composition such as, e.g. in the form of a tablet, a capsule (e.g. containing pellets which may be the same or different), sachets, powders etc

In a kit according to the invention, the composition (or part) intended for relatively fast release contains desglymidodrine as the active substance and the composition (or part) intended for controlled release contains desglymidodrine or midodrine or a combination thereof.

Interesting compositions of the invention are those, which are designed to release desglymidodrine relatively fast in order to obtain a relatively fast onset of action, i.e an action within 1-2 min after administration such as, e.g. within about 3 min. within about 4 min, within about 5 min, within about 7.5 min, within about 10 min within about 12.5 min or within about 15 min after administration.

With respect to the release of desglymidodrdne from a relatively fast onset composition, the following applies:

Tablets (plain): disintegration time less than 15 min and often less than 5 min such as, e.g. 1-3 min.

Sublingual, buccal and melt tablets: by mould technique: disintegration time less than about 30 sec such as, eg. about 2-10 sec; by compression or compacting: disintegration time less than about 4 min such as, e.g. about 2-3 min.

Other compositions normally contain desglymidodrine in dissolved form. Thus, no retardation of the release of the active drug substance from such compositions is expected.

In the following details on the controlled release compositions are given. Reference is also given to the co-pending PCT application PCT/DK01/00213 for further detail and which is incorporated herein by reference. For the sake of clarity please note that according to the discussion above, the active substance generally is desglymidodrine, but in the case of a kit midodrine may be included as well. Thus, even if e.g. only desglymidodrine is specifically mentioned, all details apply mutatis mutandi for midodrine and for a combination of desglymidodrine and midodrine. Moreover, whenever relevant, the initial relase described for the controlled release composition also applies for the desglymidodrine composition intended for a relatively fast release of the active substance.

In an embodiment of the invention, the present inventors have developed a pharmaceutical controlled release composition for oral use containing midrodrine and/or desglymidodrine and the composition is designed to the release of desglymidodrine in at least the following consecutive steps:

Step 1 an initial relatively fast release of desglymidodrine (in order to obtain a relatively fast onset of action),

Step 2 a steady release or a slower release than in step 1 of desglymidodrine (in order to maintain a plasma concentration of desglymidodrine which is prolonged and relatively constant),

Step 3 a second rise in release of desglymidodrine (in order to take advantage of absorption from the colon, i.e. such a second rise release is designed to take place when the composition (or the disintegrated parts of the composition) reaches the colon: normally this is regarded to take about 8 hours after oral intake, and

Step 4 a decline in release rate corresponding to that essentially all desglymidodrine have been released from the composition.

The first part or first composition of the kit according to the invention (i.e. the part or composition giving rise to a relatively fast onset of desglymidodrine) releases the active drug substance as described in step 1 above. Whenever relevant, details relating to such a first step including the relevant formulation techniques as well as the relevant pharmacokinetic parameters (absorption, metabolism and elimination) also apply to the non-controlled desglymidodrine composition of the invention.

The above release pattern is contemplated in order to obtain the desired plasma concentration of desglymidodrine during day and night after administration orally once or twice dally. Thus, the release pattern above is based on the following requirements with respect to the plasma concentration of desglymidodrine:

1. an initial rise in plasma concentration until a peak concentration is reached (in the present context “a peak concentration” is intended to mean a peak value, a shoulder value or a plateau value in the concentration),

2. a relatively constant plasma concentration of desglymidodrine for approximately about 4.5-14 hours such as, e.g., about 5-14 hours, about 6-14 hours, about 7-14 hours, about 8-13 hours, about 9-13 hours, about 10-14 hours, about 10-13 hours, or such as, e.g for at least about 4.5 hours, at least about 5 hours, at least about 6 hours, at least about 7 hours, at least about 8 hours, at least about 9 hours, at least about 10 hours, or at least about 11 hours. In some case, the constant plasma concentration of desglymidodrine may last for at least about 12 hours, at least about 13 hours or at least about 14 hours,

3. a decline in plasma concentration with a half-life of e.g. about 3-4 hours to avoid supine hypertension but other half-lives may also be acceptable e.g. reflecting a continous release of midodrine and/or desglymidodrine from the composition.

Compositions according to the invention are therefore designed based on the following principle; the term “part” is intended to include a separate part within the composition (the composition may contain pellets of e.g. two different types, or an integrated element of the composition, e.g. a multilayer tablet):

1. The composition contains a part intended for relatively fast release of desglymidodrine

2. The composition contains a part intended for prolonged release of desglymidodrine (or wherever relevant midodrine), and the prolonged release is intended to last for at least about 7-8 hours.

3. The composition contains a part intended to release desglymidodrine (or wherever relevant midodrine) relatively fast when the composition (or the disintegrated parts of the composition) reaches the colon, i.e. about 6-10 hours such as, e.g., about 8 hours after oral administration.

4. The release of desglymidodrine (or wherever relevant midodrine) from a composition according to the invention is terminated at the most about 12-16 hours after administration in order to obtain a wash out period during night.

In one aspect the kit according to the invention comprises a controlled release pharmaceutical composition for oral use comprising midodrine (ST 1085) or a pharmaceutically acceptable salt thereof and/or its active metabolite desglymidodrine (ST 1059) or a pharmaceutically acceptable salt thereof, the composition being adapted to release desglymidodrine in such a manner that a relatively fast peak plasma concentration of desglymidodrine is obtained and that a therapeutically effective plasma concentration of deaglymidodrine is maintained for at least about 9 hours such as, e.g. at least about 10 hours, at least about 11 hours, at least about 12 hours, at least about 13 hours, or at least about 14 hours.

More specifically, a relatively fast peak (or shoulder or plateau) plasma concentration of desglymidodrine is obtained about 15 min-6 hours such as, e,g. about 0.5-6 hours, about 1-6 hours, about 2-5.5 hours, or about 2.5-5.2 hours after oral administration of a composition according to the invention.

As mentioned above, it is important to keep the plasma concentration at a relatively constant level and, therefore, the plasma concentration of desglymidodrine after administration of midodrine and/or desglymidodrine is preferably maintained at a therapeutically active level for about 5-16 hours, such as, e.g., about 6-16 hours, about 7-16, about 8-15, about 9-15, about 10-15, about 11-14, about 12-14or about 13, or for at least about 5 hours, at least about 6 hours, at least about 7 hours, at least about 8 hours, at least about 9 hours, at least about 10 hours, at least about 11 hours, at least about 12 hours, at least about 13 hours, at least about 14 hours, at least about 15 hours or at least about 16 hours.

In the present context, the term “relatively constant level” means that n is n±60%, such as, e.g., n±50% or n±40% and wherein n is the plasma concentration in ng/ml and monitored in a healthy person. The determination of the “relatively constant level” is perfomed as described in Example 28 herein.

It should be noted that the initial fast release from the controlled release composition may be supplemented with or replaced by a separate fast onset composition (e.g another composition or another part or composition of the kit) resulting in a peak plasma concentration within the period stated for the initial rise in plasma concentration. A separate fast onset composition gives a flexibility with respect to the dose administered, i.e. if needed a relatively low or a relatively high dose of the active drug substance may be administered dependent on the patient's needs. Thus, in other aspects the invention relates to such relatively fast onset compositions.

In principle, relevant active drug substances for use in a composition according to the invention are any drug substance for which a dissolution pattern as described below is of relevance. The most interesting drug substances in this respect and with respect to treatment of orthostatic hypotension and urinary incontinence are the prodrug midodrine and its active metabolite desglymidodrine. In a preferred aspect, a composition according to the invention includes desglymidodrine, or a combination of midodrine and deaglymidodrine. Of course such compositions may also contain other active drug substances, if relevant.

Generally, after oral administration of a composition according to the invention containing desglymidodrine, a peak plasma concentration of desglymidodrine is obtained abot 1-2 min after administration such as, eg. about 2-5 min, about 5-10 min or about 15-30 min after administration or 15-90 min after oral administration. Moreover, the plasma concentration of desglymidodrine after oral administration is maintained at a relatively constant level for about 0.7-4 hours such as, e.g at least about 0.7 hours, at least about 1 hour, at least about 2 hours, at least about 3 hours, or at least about 4 hours.

To this end, the tern “relatively constant” is intended to mean m is m±60%, such as, e.g., m±50% or m±40% and wherein m is the plasma concentration in ng/ml and monitored in a healthy person. The determination of the “relatively constant level” is perfomed as described in Example 28 herein.

In the following further details on a controlled release composition according to the invention and, accordingly, also on a controlled release composition of a kit according to the invention is given.

Examples on compositions (inter alia relatively fast onset compositions and controlled release compositions, respectively) are illustrated in the Examples.

Dissolution Requirements

As described in the following, a target plasma profile and release profile can be designed for the controlled release composition or the controlled release part of the kit comprising desglymidodrine.

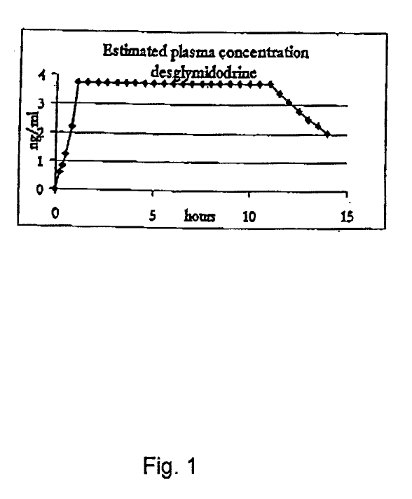

i) Based on the desired effect of desglymidodrine and plasma profiles of desglymidodrine after administration of midodrine a target in vivo profile has been estimated (FIG. 1) (see also PCT application No. PCT/DK01/00213 or PCT/DK01/00214 for further details).

Target release in vitro profile for a controlled release composition estimated as described above:

| Time (hours) | % w/w released desglymidodrine |
|---|---|
| 0.5 | 25 |
| 1 | 35 |
| 2 | 39 |
| 3 | 47 |
| 4 | 53 |
| 6 | 60 |
| 6 | 66 |
| 7 | 73 |
| 8 | 80 |
| 9 | 87 |
| 10 | 93 |
| 12 | 100 |

In order to reflect the second rise in release of desglymidodrine corresponding to the time when the composition reaches the colon, the following target profile is also relevant:

| Time (hours) | % w/w released desglymidodrine |
|---|---|
| 0.5 | 25 |
| 1 | 35 |
| 2 | 39 |
| 3 | 47 |
| 4 | 53 |
| 5 | 60 |
| 6 | 66 |
| 7 | 75 |
| 8 | 90 |
| 9 | 95 |
| 10 | 97 |
| 11 | 99 |
| 12 | 100 |

As apparent from the above, an initial relatively fast release of desglyrnidodrine is suitable and after about 6-8 hours a second rise in release should be observed. Accordingly, a target release rate profile is as follow (the release rate is given in % dissolved/hour).

about 35%/hour about 30 min after start of the dissolution test,

about 12%/hour about 1 hour after start of the dissolution test,

about 6%/hour about 2 hours after start of the dissolution test,

about 7%/hour about 3 hours after start of the dissolution test,

about 6.5%/hour about 4 hours after start of the dissolution test,

about 6.5%/hour about 5 hours after start of the dissolution test,

about 7.5%/hour about 6 hours after start of the dissolution test,

about 12%/hour about 7 hours after start of the dissolution test,

about 10%/hour about 8 hours after start of the dissolution test,

about 3.5%/hour about 9 hours after start of the dissolution test

about 2%/hour about 10 hours after start of the dissolution test,

about 1%/hour about 12 hours after start of the dissolution test.

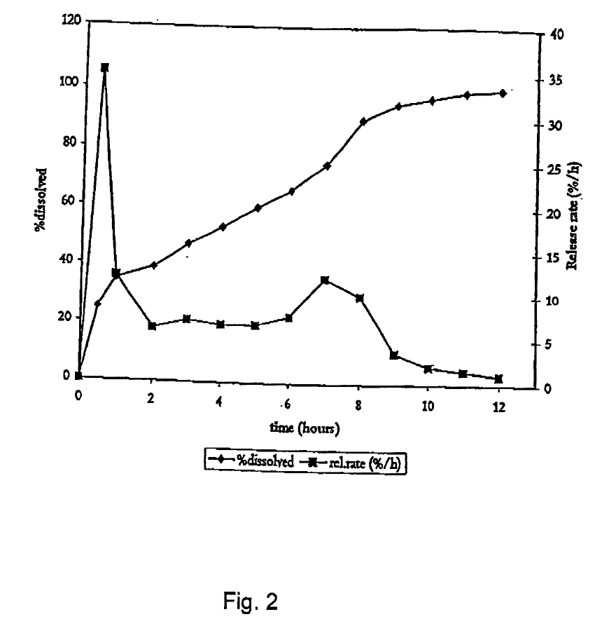

In FIG. 2 is given a target dissolution profile and a target release rate curve. Suitable dissolution methods are found in the Examples herein.

Specific embodiments of interest are as follows (wherein the release pattern of desglymidodrine (or whenever relevant midodrine) from the controlled release composition—when tested in vitro using a Dissolution Method described in the Experimental part herein—is).

| | | | Diss. Method^{a} | | | | | | |
|---|---|---|---|---|---|---|---|---|---|
| | | Diss. Method | Diss. Method | I, II, III or IV | Diss. Method | Diss. Method | Diss. Method^{b} | | |
| Time | | I or II (% w/w) | III or IV (% w/w) | (% w/w) | I, II, III or IV (% w/w) | I, II, III or IV (% w/w) | | | |
| ≦30 | min. | 1-15% | 1-15% | | 1-15% | 1-15% | | | |
| 30 | min. | 10-35% | 10-35% | 25% | 10-35% | 15-35% | 25% | 28% | 20% |
| 1 | h | 15-40% | 15-40% | 35% | 15-40% | 20-40% | 35% | 35% | 20% |
| 2 | h | 20-50% | 20-50% | 39% | 20-50% | 25-50% | 39% | 41% | 20% |
| 3 | h | 20-55% | 20-55% | 47% | 20-55% | 30-55% | 47% | 45% | 20% |
| 4 | h | 25-75% | 25-75% | 53-56% | 25-75% | 53% | 55% | 25% | |
| | | (25-65%) | (25-65%) | | (25-65%) | (40-65%) | | | |
| 6 | h | 30-74% | 30-74% | 68-72% | 30-74% | 60-74% | 66% | 70% | 45% |
| 7 | h | | | | 35-85% | 60-85% | 75% | 78% | 75% |
| 8 | h | 40-85% | 40-95% | 80-85% | 45-95% | 70-95% | 80% | 90% | 90% |
| | | | (45-85%) | | | | | | |
| 10 | h | 65-100% | 65-100% | 93% | 65-100% | 80-100% | 90% | 95% | 95% |
| 12 | h | 90-110% | 75-110% | 100% | 90-110% | 90-110% | 100% | 100% | 100% |

^{a}±30% such as, e.g. ±25%, ±20%, ±15%, ±10% or ±0%
^{b}±30% w/w, ±20% w/w, ±10% w/w, ±7.5% w/w, ±5% w/w, ±0% w/w

In those cases where the controlled release composition of a kit according to the invention contains midodrine or a pharmaceutically acceptable salt thereof then the release pattern of midodrine generally follows the patterns given above for desglymidodrine.

If the controlled release composition of a kit according to the invention contains midodrine or a pharmaceutically acceptable salt thereof and desglymidodrine or a pharmaceutically acceptable salt thereof, then the release pattern of the sum of midodrine and desglymidodrine is calculated on a molar basis follows the patterns given above for midodrine.

As discussed earlier the release rate of desglymidodrine is important in order to achieve a suitable release pattern. Thus, a controlled release composition according to the present invention normally has a release rate of desglymidodrine (or whenever relevant midodrine)—when tested in vitro employing any of Dissolution Method I, II, III or IV—that corresponds to a curve that has a shape corresponding to

i) a relatively fast first initial release followed by

ii) a steady release or a slower release than in step i) above, which is followed by

iii) a second rise in release rate and, finally,

iv) a decline in release rate.

In general, the second rise in release rate takes place 5-10 hours such as, e.g., about 5-9 hours, about 6-8 hours after start of the dissolution test, or 6.5-9 hours after start of the dissolution test simulating the time it takes to reach the colon after oral administration.

With respect to the steady release period, it normally starts about 1-3 hours after the start of the dissolution test, and the steady release is maintained for at least 2 hours such as, e.g. at least 3 hours, at least 4 hours, at least 6 hours, at least 6 hours, at least 7 hours, at least 8 hours such as about 6-8 hours.

More specifically, the release rate of desglymidodrine (or midodrine or the sum of midodrine and desglymidodrine on a molar basis) from a controlled release composition of a kit according to the invention—when tested in vitro employing dissolution apparatus 2 (paddle) according to USP and Ph. Eur. 100 rpm, 0.1 N hydrochloric acid as dissolution medium or any of Dissolution Method I, II, III or IV as described herein and a temperature of 37° C.—in %/hour is as follows (±10-40% such as, e.g. ±10-30% or ±10%, ±15% or ±20% of the values stated below):

about 35%/hour about 30 min after start of the test (range e.g. 15-40%/hour),

about 12%/hour about 1 hour after start of the test (range e.g 4-15%/hour),

about 6%/hour about 2 hours after start of the test (range e.g. 2-10%/hour),

about 7%/hour about 3 hours after start of the test (range e.g. 2-10%/hour),

about 6.5%/hour about 4 hours after start of the test (range e.g. 2-15%/hour),

about 7.5%/hour about 6 hours after start of the test (range e.g. 2-30% such as, e.g., 2-10%/hour),

about 10%/hour about 8 hours after start of the test (range e.g. 2-15%/hour),

about 2%/hour about 10 hours after start of the test (range e.g. 0-10%/hour),

about 1%/hour about 12 hours after start of the test (range e.g. 0-10%/hour).

A controlled release composition according to the invention is normally suitable for administration once or twice daily, and it differs from a plain tablet composition, e.g. Gutron® tablets, in many ways. In the following is given pharmacokinetic values of importance for achievement of a prolonged therapeutic effect of a composition according to the invention. Further details concerning the definition of the parameters and the method of obtaining relevant values are given in Example 28 herein.

When tested as described in Example 28 herein, W₅₀ of desglymidodrine (defined as corresponding to the time the plasma concentration curve is or is above 50% of the Cₘₐₓ value) is from about 5 to about 12 hours such as, e.g. from about 6 to about 11 hours such as, e.g. at least about 7 hours.

Furthermore, Tₘₐₓ is increased with a factor of at least 2 when compared with a plain Gutron® tablet administered in the same dose. Tₘₐₓ is determined from a plasma concentration versus time curve and the plasma concentration reflects the sum concentration in nmol/l of midodrine and desglymidodrine.

MRT (mean residence time) is increased with a factor of at least 1.5 such as, e.g., at least 2, at least 2.5 or at least 3 when compared with a plain Gutron® tablet administered in the same dose. MRT is determined from a plasma concentration versus time curve and the plasma concentration reflects the sum concentration in nmol/l of midodrine and desglymidodrine.

MRT for midodrine is at least about 1.5 hours such as, e.g., at least about 2 hours, at least about 2.5 hours or at least about 3 hours, and/or MRT for desglymidodrine is at least about 6 hours such as, e.g., at least about 7 hours, at least about 7.5 hours, at least about 8 hours, at least about 8.5 hours, at least about 9 hours, or at least about 9.5 hours.

The invention is further illustrated in the drawing, wherein

FIG. 1 shows the estimated plasma concentration of desglymidodrine,

FIG. 2 shows the estimated in vitro target for dissolution of desglymidodrine and the estimated release rate, and

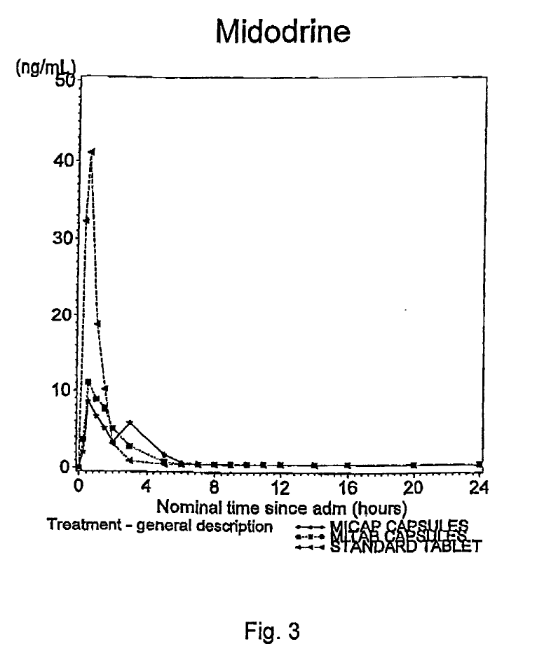

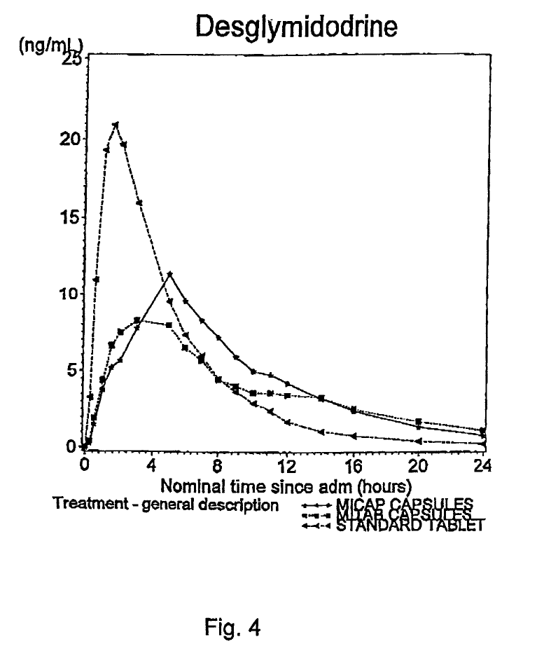

FIGS. 3-4 illustrate the results of Example 28.

The following examples are intended to illustrate specific embodiments of the present invention but are not intended in any way to limit the invention. Some of the examples are included in order to illustrate that the release rate and dissolution characteristics of a composition can be changed by varying a number of formulation parameters.

### METHODS

### Dissolution Method I

| | |
|---|---|
| Apparatus | Ph.Eur/USP dissolution |
| | apparatus + Perkin |
| | Elmer fully automatic dissolution |
| | system + Disslab PC-programme |
| Glass fibre filter | 0.7 μm |
| Dissolution medium | 600 ml 0.1N HCl |
| Rotation speed | 100 rpm |
| Stirrer | Basket |
| Sampling times | As appear from tables |
| Detection wavelength | 290 nm |
| Measuring equipment | UV-spectrophotometer, |
| | 10 mm quartz cuvette |
| Temperature of dissolution medium | 37° C. ± 0.5° C. |

Reagents:

0.1N HCl is prepared by dilution of concentrated HCl (37%) with purified water.

Standards:

Two solutions are prepared with a concentration of 10 μg/ml midodrine hydrochloride in 0.1 N HCl. 0.1 N HCl is used as blind. The absorbances of the solutions are measured on the spectrophotometer.

E_{1%/cm} is calculated.

E_{1%/cm}=A×1000, where 1000 is due to the fact, that the solution is only 0.001%.

The mean value of the to measurements is inserted in the software programme in accordance with the manual for the automated dissolution system.

Performance:

600 ml 0.1N HCl is filled in each of the six vessels in the dissolution equipment. The media is heated to a temperature of 37° C.±0.5° C. One weighed tablet is placed in each of the six baskets. The stirring is started as soon as the baskets are lowered into the vessels. The sample is filtered through a 0.7 μm filter. The absorbances of the filtered samples are measured directly at 290 nm.

### Dissolution Method II, HPLC-Detection

The dissolution parameters are described in Method I. The measurement is performed by HPLC.

| | |
|---|---|
| Column | Spherisorb ODS-1; 5 μm; 25 cm; ID 4.6 μm |
| Injection volume | 20 μl |
| Flowrate | 1.0 ml/min |
| Mobile phase | Phosphate buffer pH 3: methanol 77:23 (v/v) |
| Detection | 290 nm |
| Runtime | 30 min |

Reagents for dissolution are described in Method I.

Buffer solution pH 3 is prepared by dissolving 23.6 g potassium dihydrogen phosphate in 900 ml purified water. o-Phosphoric acid 85% is used to adjust pH. The flask is filled to 1 l.

Standards:

Two stock Solutions with a concentration of midodrine hydrochloride 120 μg/ml in 0.01 N HCl are prepared. The solutions are stored in refrigerator.

Prepare from each stock solution two standard solutions with a concentration of midodrine hydrochloride approximately 1.5 μgl/ml and 15 μg/ml respectively, diluted with 0.01N HCl.

Desglymidodrine hydrochloride is quantified against the standard curve of midodrine hydrochloride. The relative response factor is 1.25 for Desglymidodrine hydrochloride to midodrine hydrochloride.

Performance:

The dissolution is performed as described in Method I. The sample is withdrawn with a pipette and transferred to a syringe. The sample is filtered through a 0.7 μm filter. The first ml is returned to the vessel in order to reduce deviation from the desired volume. A sample of approximately 1.5-2 ml is transferred to the vial the rest is returned to the vessel. The absorbances of the filtered samples are measured as described.

Calculations:

A standard curve is calculated by linear regression, using the standard solutions. The peak area of the sample is the sum of the peak area of Midodrine Hydrochloride and the peak area of Desglymidodrine hydrochloride, where the latter is divided by the relative response factor 1.25.

The results are calculated as % released at any time and presented as a mean value of the six samples together with min and max.

$$\text{\% dissolved} \frac{A vol 100}{b x}$$

Where

A sum of peak area of midodrine hydrochloride and Desglymidodrine hydrochloride (corr.)

Vol 600 ml for tablets (MITAB)

100%

b slope of the calibration curve (A/mg/ml)

x declared amount (mg)

### Dissolution Method III

| | |
|---|---|
| Apparatus | Ph.Eur/USP dissolution apparatus + Perkin Elmer |
| | fully automatic dissolution system + Disslab PC- |
| | programme |
| Glass fibre filter | 0.7 μm |
| Dissolution medium | 600 ml 0.1N HCl |
| at the beginning | |
| Dissolution media at | Addition of 130 ml 0.23 M Na₃PO₄ solution |
| change to pH 6.0 | |
| Dissolution media at | Addition of further 70 ml 0.23 M Na₃PO₄ solution |
| change to pH 7.5 | |
| Time for change | 2 hours (120 min) |
| to pH 8.0 | |
| Time for change | 7.5 hours (450 min) |
| to pH 7.5 | |
| Rotation speed | 100 rpm |
| Stirrer | Basket |
| Sampling times | As appear from tables |
| Detection wavelength | 290 nm |
| Measuring equipment | uv-spectrophotometer, 10 mm quartz cuvette |
| Temperature of | 37° C. ± 0.5° C. |
| dissolution medium | |
| Reference | An empty capsule dissolved in 600 ml 0.1 N HCl |
| (vessel no 7) | |
| Vessel no 7 is added | |
| Na₃PO₄ solution in | |
| parallel with the six | |
| sample vessels | |

Reagents:

0.1 N HCl is prepared by dilution of concentrated HCl (37%) with purified water.

0.23 M Na₃PO₄ solution: Dissolve an amount of Na₃PO₄.12H₂O in a bit of 1M HCl-R and add water to a concentration of 0.23 M. (Strong alkaline).

Buffer solution pH 6.0: 600 ml 0.1 N HCl is added 130 ml 0.23 M Na₃PO solution.

Buffer solution pH 7.5; Buffer solution pH 6.0 is added further 70 ml 0.23 M Na₃PO solution.

Standards:

Two solutions are prepared with a concentration of 10 μg/ml midodrine hydrochloride in 0.1 N HCl. 0.1N HCl is used as blind. The absorbances of the solutions are measured on the spectrophotometer.

E_{1%/cm} is calculated. It has previously been determined, that the E_{1%/cm} is the same for the three media, so it is only necessary to perform the test in 0.1 N HCl.

E_{1%/cm}A×1000, where 1000 is due to the fact, that the solution is only 0.001%.

The mean value of the to measurements is inserted in the software programme in accordance with the manual for the automated dissolution system.

Performance:

600 ml 0.1N HCl is filled in each of the seven vessels in the dissolution equipment. The media is heated to a temperature of 37° C.±0.5° C. One weighed capsule is placed in each of the six baskets. In the seventh basket an empty capsule is placed. This is measured as a blank reference. The stirng is started as soon as the baskets are lowered into the vessels. The measured amounts of buffer solution, needed for the changes of pH in the vessels, are preheated to 37° C., before addition to the vessels. When the buffer is to be added, the baskets are elevated from the vessels, the buffer is added, the solution in the vessel is stirred to homogenise the solution and the baskets are lowered into the vessels again. The sample is filtered through a 0.7 μm filter. The absorbances of the filtered samples are measured directly at 290 nm.

### Dissolution Method IV, HPLC-Detection

The dissolution parameters are described in Method III. The measurement is performed by HPLC.

| | |
|---|---|
| Column | Spherisorb ODS-1; 5 μm; 25 cm; ID 4.5 μm |
| Injection volume | 20 μl |
| Flowrate | 1.0 ml/min |
| Mobile phase | Phosphate buffer pH 3: methanol 77:23 (v/v) |
| Detection | 290 nm |
| Runtime | 30 min |

Reagents for dissolution are described in Method III.

Buffer solution pH 3 is prepared by dissolvng 23.6 g potassium dihydrogen phosphate in 900 ml purified water. o-Phosphoric acd 85% is used to adjust pH. The flask is filled to 1 l.

Standards:

Two stock solutions with a concentration of midodrine hydrochloride 120 μg/ml in 0.01N HCl are prepared. The solutions should be stored in refrigerator.

Prepare from each stock solution two standard solutions with a concentration of midodrine hydrochloride approximately 15 μg/ml and 15 μg/ml respectively, diluted with 0.01N HCl.

Desglymidodrine hydrochloride is quantified against the standard curve of midodrine hydrochloride. The relative response factor is 1.25 for desglymidodrine hydrochloride to midodrine hydrochloride.

Performance:

The dissolution is performed as described in Method III. The sample is withdrawn with a pipette, transferred to a syringe. The sample is filtered through a 0.7 μm filter. The first ml is returned to the vessel in order to reduce deviation from the desired volume. A sample of approximately 1.5-2 ml is transferred to the vial, the rest is returned to the vessel. The absorbances of the filtered samples are measured as described.

Calculations:

A standard curve is calculated by linear regression, using the standard solutions. The peak area of the sample is the sum of the peak area of midodrine hydrochloride and the peak area of desglymidodrine hydrochloride, where the latter is divided by the relative response factor 1.25.

The results are calculated as % released at any time and presented as a mean value of the six samples together with min and max.

$$\text{\% dissolved} \frac{A vol 100}{b x}$$

Where

A sum of peak area of midodrine hydrochloride and desglymidodrine hydrochloride (corr.)

Vol 600 ml for up to 2 hours 730 ml for up to 7.5 hours and 800 ml for up to 12 hours for capsules (MICAP)

100%

b slope of the calibration curve (A/mg/ml)

x declared amount (mg)

### Dissolution Method V (Melt Tablets)

Dissolution apparatus 2 according to USP and Ph. Eur.

Paddle method

Rotations: 50 rpm

Temperature: 37° C.

Dissolution medium: isotonic buffer pH 6.8 or purified water

Volumen: 600 ml

Sample time: 5 min

RP-HPLC: Phosphate buffer pH 3; methanol 77:23 (v/v)

Detection at 290 nm.

### Dissolution Metod VI (Sublingual Tablets)

Dissolution apparatus 2 according to USP and Ph. Eur.

Paddle method

Rotations: 50 rpm

Temperature: 37° C.

Dissolution medium: isotonic buffer pH 6.8 or purified water

Volume; 600 ml

Sampling time: 5 min

RP-HPLC: Phosphate buffer pH 3: methanol 77:23 (v/v)

Detection at 290 nm.

### EXAMPLES

In the following examples desglymidodrine refers to desglymidodrine-HCl, if nothing else is mentioned.

### Example 1

### Preparation of a Desglymidodrine Composition by Employment of Triple Compression

The following example illustrates a composition made by employment of triple compression

Composition:

Core:

| | | |
|---|---|---|
| Desglymidodrine | 4.0 | mg |
| Klucel MF | 2.0 | mg |
| Methocel E 50 | 94.0 | mg |
| 1^{st} compression layer: | | |
| Desglmidodrine | 1.2 | mg |
| Klucel MF | 6.6 | mg |
| Methocel E 15 | 157.2 | mg |
| 2^{nd} compression layer | | |
| Desglymidodrine | 2.3 | mg |
| Methocel E 50 | 247.7 | mg |

Using the core composition a core weighing 100 mg is compressed using a punch 6 mm in diameter. The core is compression coated using 165 mg of the 1^{st} compression layer composition and a punch of 9 mm in diameter. The thus compression coated core is compression coated again using 250 mg of the 2^{nd} compression layer composition and a punch of 11 mm in diameter.

A composition comprising desglymidodrine 0.96 mg, Methocel E5 9.7 mg and talc 8.7 mg is applied to the tablet by spray coating.

### Example 2

### Preparation of a Desglymidodrine Composition by Employment of Triple Compression

The following example illustrates a composition made by employment of triple compression

Composition:

| | | |
|---|---|---|
| Core: | Desglymidodrine | 1.33 mg |
| | Hydroxypropylmethyl cellulose E 50 | 48.67 mg |
| | Croscamellose sodium | 10.00 mg |
| | | 60.00 mg |
| 1^{st} compression layer | Desglymidodrine | 0.50 mg |
| | Hydroxypropylmethyl cellulose E 15 | 126.50 mg |
| | Hydroxypropylmethyl cellulose | 8.00 mg |
| | K 100 LV | 135.00 mg |
| 2^{nd} compression layer | Desglymidodrine | 1.60 mg |
| | Hydroxypropylmethyl cellulose E 50 | 143.40 mg |
| | | 145.00 mg |

Using the core composition a core weighing 60 mg is compressed using a punch of 6 mm in diameter. The core is compression coated using 135 mg of the 1^{st} compression layer composition and a punch of 9 mm in diameter. The thus compression coated core is compression coated again using 145 mg of the 2^{nd} compression layer composition and a punch of 11 mm in diameter,

A composition comprising desglymidodrine 0.59 mg, hydroxypropylmethyl cellulose E 5 3.58 mg, talc 2.65 mg and propylene glycol 0.71 mg is applied to the tablet by spray coating.

Finally a top coat comprising hydroxypropylmethyl cellulose E 5 1.79 mg, talc 1.25 mg and propylene glycol 0.36 mg is applied to the tablet by spray coating.

### Example 3

### Desglymidodrine Composition Made as a Coated Matrix

The following compositions are prepared:

| Composition 1; | Core: | | |
|---|---|---|---|
| | Desglymidodrine | 8.0 | mg |
| | Klucel LF | 342.0 | mg |
| | Insoluble inner coat | | |
| | Methocel E 5 | 0.2 | mg |
| | Magnesium stearate | 0.1 | mg |
| | Talc Ponderax | 0.4 | mg |
| | Anti foam | 4.8 | μg |
| | Eudragit NE 30 D | 4.5 | mg |
| | Soluble outer coat | | |
| | Methocel E 5 | 1.8 | mg |
| | Talc Ponderax | 1.8 | mg |
| Composition 2: | Core: | | |
| | Desglymidodrine | 8.0 | mg |
| | Klucel MF | 342.0 | mg |
| | Insoluble inner coat | | |
| | Methocel E 5 | 0.2 | mg |
| | Magnesium stearate | 0.1 | mg |
| | Talc Ponderax | 0.4 | mg |
| | Anti foam | 4.8 | μg |
| | Eudragit NE 30 D | 4.5 | mg |
| | Soluble outer coat | | |
| | Methocel E 5 | 1.8 | mg |
| | Talc ponderax | 1.8 | mg |

Cores of both composition 1 and composition 2 are compressed using a punch 10 mm in diameter. Core weighing 350 mg.

Both types of cores are coated with an insoluble inner coat and a soluble outercoat. The release profile can be shifted up or down by changing the amount of weight increase of cores when applying the inner coat.

If suitable, the release profile can be changed by coating with other acrylic resins such as Eudragit RL 30 D, Eudragit RS 30 D or combinations thereof, or using other types of film forming agents such as ethylcellulose or silicone polymers. Furthermore, the release profile can be changed by using other types of matrix former such as acrylic resins, other types of cellulose ethers such as L-HPC (low-substituted hydroxypropylcellulose), HPC (hydroxypropylcellulose), HPMC (hydroxypropylmethylcellulose), HEC (hydroxyethylcellulose), MC (methylcellulose), HEMC (hydroxyethylmethylcellulose), EC (ethylcellulose) or other viscosity grades of HPC (hydroxypropylcellulose).

### Example 4

### Multilayer Coating Compositions

Composition:

| | | | |
|---|---|---|---|
| Composition 1: | Core (Non parell) | 200 | mg |
| | 1. coat | | |
| | Desglymidodrine | 3.2 | mg |
| | Methocel E 5 M | 0.3 | mg |
| | Magnesium Stearate | 60.0 | μg |
| | Talc ponderax | 0.5 | mg |
| | Anti foam | 4.0 | μg |
| | Eudragit NE 30 D | 5.2 | mg |
| | 2. coat | | |
| | Desglymidodrine | 2.4 | mg |
| | Methocel E 5 M | 0.3 | mg |
| | Magnesium Stearate | 60.0 | μg |
| | Talc ponderax | 0.5 | mg |
| | Anti foam | 4.0 | μg |
| | Eudragit NE 30 D | 8.1 | mg |
| | 3. coat | | |
| | Desglymidodrine | 1.6 | mg |
| | Methocel E 5 M | 0.3 | mg |
| | Magnesium Stearate | 80.0 | μg |
| | Talc ponderax | 0.6 | mg |
| | Anti foam | 6.0 | μg |
| | Eudragit NE 30 D | 7.1 | mg |
| | 4. coat | | |
| | Desglymidodrine | 0.8 | mg |
| | Methocel E 5 M | 0.4 | mg |
| | Magnesium Stearate | 80.0 | μg |
| | Talc ponderax | 0.7 | mg |
| | Anti foam | 6.0 | μg |
| | Eudragit NE 30 D | 7.8 | mg |
| | Outer coat | | |
| | Methocel E 5 | 1.0 | mg |
| | Talc ponderax | 1.0 | mg |

Non-pareil beads are coated in four steps with four different films in a fluid bed coater.

1. film comprising 1. coat

2. film comprising 2. coat

3. film comprising 3. coat

4. film comprising 4. coat.

A final layer of coating comprising the outer coat is applied and the films are cured at 70° C.

| | | |
|---|---|---|
| Composition 2: | Core (Non pareil) | 200 mg |

Non-pareil beads are coated in seven steps with four different films aternating with a blank film in a fluid bed coater.

The four different film formulations are similar to the four different film formulations in composition 1, the alternating coats are as follows:

Alternating Coat

| | | |
|---|---|---|
| Methocel E 5 M | 0.2 | mg |
| Magnesium stearate | 40.0 | μg |
| Talc ponderax | 0.3 | mg |
| Anti foam | 2.0 | μg |
| Eudragit NE 30 D | 3.5 | mg |

1. film comprising 1. coat

2. film comprising Alternating coat

3. film comprising 2. coat

4. film comprising Alternating coat

5. film comprising 3. coat

6. film comprising Alternating coat

7. film comprising 4. coat

A final layer of coating comprising outer coat in composition 1 is applied and the films are cured at 70°C.

| | | | |
|---|---|---|---|
| Composition 3: | Core (Non pareil) | 200 | mg |
| | 1. cost | | |
| | Desglymidodrine | 3.2 | mg |
| | Paraffin, solid | 0.3 | mg |
| | Acetyltributyl citrate | 0.1 | mg |
| | Ethylcellulose | 1.9 | mg |
| | Aerosil 200 | 28.0 | μg |
| | 2. coat | | |
| | Desglymidodrine | 2.4 | mg |
| | Paraffin, solid | 0,3 | mg |
| | Acetyltributyl citrate | 0.1 | mg |
| | Ethylcellulose | 2.2 | mg |
| | Aerosll 200 | 32.0 | μg |
| | 3. coat | | |
| | Desglymidodrine | 1.6 | mg |
| | Paraffin, solid | 0.4 | mg |
| | Acetyltributyl citrate | 0.1 | mg |
| | Fthylceliulose | 2.5 | mg |
| | Aerosil 200 | 40.0 | μg |
| | 4. coat | | |
| | Desglymidodrine | 0.8 | mg |
| | Paraffin, solid | 0.4 | mg |
| | Acetyltributyl citrate | 0.2 | mg |
| | Ethylcellulose | 2.5 | mg |
| | Aerosil 200 | 40.0 | μg |
| | Outer coat | | |
| | Paraffin, solid | 0.5 | mg |
| | Acetyltributyl citrate | 0.2 | mg |
| | Ethylcellulose | 3.3 | mg |
| | Aerosil 200 | 50.0 | μg |

Non-pareil beads are coated in four steps with four different films in a fluid bed coater:

1. film comprising 1. coat

2. film comprising 2. coat

3. film comprising 3. coat

4. film comprising 4. coat.

A final layer of coating comprising outer coat is applied.

If suitable, the release profile can be changed by coating with other acrylic resins such as Eudragit RL 30 D, Eudragit RS 30 D or combinations thereof, or using other types of film forming agents such as ethylcellulose or silicone polymers, or incorporating lipophilic compounds such as, e.g., stearic acid, capric acid or hydrogenated castor oil in the film.

### Example 5

### Preparation of a Controlled Release Composition Using Commercially Available Filmforming Agents

The present example illustrates the preparation of a coated pellet composition. The aim is to prepare pellets having a release kinetic different from zero order release.

Pellets are prepared from the following ingredients:

| | | |
|---|---|---|
| I | Desglymidodrine | 482.3 g |
| II | Microcrystalline cellulose (Type PH 101) | 778.0 g |
| III | Lactose monohydrate | 2700.0 g |
| IV | Sodium carboxymethylcellulose | 40.0 g |
| V | Purified water | 1120.0 g |

I+II+III+IV are admixed in a Fielder intensive mixer at an appropriate time and mixing intensity.

V is applied to the mixture (I-IV) while mixing. When V is applied the mixing is continued at an appropriate time with an appropriate mixing intensity.

The wetted mass is extruded through a screen with apertures between 0.4-1.0 mm.

The extrudate is spheronised until the surface of the resulting pellets is smooth.

An inner and an outer coating are applied:

Inner Coat

The weight of the pellets is increased with 8.5% w/w.

| | | |
|---|---|---|
| I | Hydroxypropylmethylcellulose | 13.5 g |
| II | Magnesium stearate | 2.9 g |
| II | Talc | 25.2 g |
| IV | Eudragit NE 30 D | 895.1 g |
| V | Purified water | 1135.4 g |

The pellets are coated in a fluid bed with appropriate process parameters.

lmmediately after the inner coat has been applied an outer coat is applied.

Outer Coat

The weight of the pellets is increased with 1% w/w.

| | | |
|---|---|---|
| I | Hydroxypropylmethylcellulose | 20.0 g |
| II | Talc | 20.0 g |
| III | Purified water | 460.0 g |

The pellets are coated in a fluid bed with appropriate process parameters.

The weight of 1 unit dose containing 30 mg desglymidodrine is 272.7 mg.

The release profile can be shifted up or down by changing the amount of weight increase of pellets when applying the inner coat.

The release profile may be changed using different mixtures of pellet fractions having different amounts of inner coating applied. The release profile may also be changed by coating with other acrylic resins such as Eudragit RL 30 D, Eudragit RS 30 D or combinations thereof, or using other types of film forming agents such as ethylcellulose or silicone polymers.

Furthermore, the release profile can be changed by applying a fraction of non-coated pellets or by applying an enteric coating to a fraction of pellets.

### Example 6

### Preparation of a Controlled Release Composition Using a Film Containing Paraffin

The present example illustrates the preparation of a coated pellet composition. The aim is to prepare pellets having a release kinetic diffrent from zero order release.

Coated pellets are prepared from the following ingredients;

The composition and manufacturing process of pellets are similar to Example 5.

A paraffin-containing coating are applied; the weight of the pellets is increased with 6% w/w.

| | | | |
|---|---|---|---|
| I | Paraffin, solid | 29.89 | g |
| II | Acetyltributyl citrate | 10.53 | g |
| III | Ethyl cellulose | 196.61 | g |
| IV | Silicium dioxide (Aerosil 200) | 2.95 | g |
| V | Isopropyl alcohol | 3970.03 | g |

The pellets are coated in a fluid bed with appropriate process parameters.

The weight of 1 unit dose containing 30 mg desglymidodrine is 263.7 mg.

### Example 7

### Preparation of a Controlled Release Composition Having a Zero Order Release

The present example illustrates the preparation of a coated bead composition. The aim is to prepare beads having a zero order release kinetic.

Coated beads are prepared as follows:

Non dissolvable non-pareil beads of equal size are coated with a suspension of desglymidodrine. A diffusion barrer is coated on top of the desglymidodrine layer, and thereby controlling the release of desglymidodrine.

4000 g non-pareil beads having a uniform particle see in a range between 0.4 mm and 1.0 mm are transferred to a fluid bed coater.

The beads are coated with coating suspension 1 (containing desglymidodrine)

| | | | |
|---|---|---|---|
| I | Hydroxypropylmethylcellulose | 8.8 | g |
| II | Magnesium stearate | 1.9 | g |
| III | Talc | 16.5 | g |
| IV | Eudragit NE 30 D | 585.1 | g |
| V | Purified water | 742.1 | g |
| VI | Desglymidodrine | 160.7 | g |

The weight of the beads is increased with 9% w/w.

The beads are coated employing appropriate process parameters.

Immediately after coating with suspension 1 a second coating suspension is applied.

The beads are coated with coating suspension 2:

| | | | |
|---|---|---|---|
| I | Hydroxypropylmethylcellulose | 11.7 | g |
| II | Magnesium stearate | 2.5 | g |
| III | Talc | 21.7 | g |
| IV | Eudragit NE 30 D | 772.3 | g |
| V | Purified water | 979.6 | g |

The weight of the coated beads is increased with 6% w/w.

The pellets are coated employing appropriate process parameters.

Immediately after coating with suspension 2 a third coating suspension is applied.

The beads are coated with coating suspension 3:

| | | | |
|---|---|---|---|
| I | Hydroxypropylmethylcellulose | 23.3 | g |
| II | Talc | 23.3 | g |
| III | Purified water | 536.8 | g |

The weight of the coated beads is increased with 1% w/w.

The beads are coated in a fluid bed employing appropriate process parameters.

The weight of 1 unit dose containing 20 mg desglymidodrine is 587.5 mg.

By changing the weight gain of the beads when applying the second coating suspension, the release profile can be shifted up or down.

The release profile may be changed using different mixtures of bead fractions having different amounts of second coating suspension applied. The release profile may also be changed by coating with other acrylic resins such as Eudragit RL 30 D, Eudragit RS 30 D or combinations thereof, or using other types of film forming agents such as ethylcellulose or silicone polymers.

The above-mentioned filmforming agents can also be combined with pore forming agents such as cellulose ethers, polyoles, PEG's.

Furthermore, the release profile can be changed by applying an enteric coating to a fraction of the coated beads.

### Example 8

### Preparation of a Zero Order Controlled Release Composition

The present example illustrates the preparation of a coated minitablet composition. The aim is to prepare coated minitablets of equal size in order to obtain a zero order release kinetic.

Formulation of minitablets:

| | | | |
|---|---|---|---|
| I | Desglymidodrine | 800.0 | g |
| II | Dicalcium phosphate | 2960.0 | g |
| III | Talc | 100.0 | g |
| IV | Magnesium stearate | 40.0 | g |
| V | Polyvinylpyrrolidone 90 | 100.0 | g |
| VI | Purified water | 800.0 | g |

V is dissolved in VI.

I+II are transferred to a Fielder intensive mixer and admixed at an appropriate time and mixing intensity.

The mixture is wetted with the solution V+VI.

Granulation is performed at an appropriate time and mixing intensity.

The drying of the wet granulate is carried out in an Aeromatic fluid bed.

The dried granulate is passed through a suitable sieve IV+V are sieved through a 0.3 mm sieve and admixed to the sieved particulate mixture in a cube mixer for 10 min.

The thus obtained particulate mixture is compressed into tablets weighing 15 mg.

A dose of 30 mg desglymidodrine corresponds to 10 minitablets.

Coating of the Minitablets:

The minitablets are coated with inner and outer coatings corresponding to the description of coating suspension 2 and 3 in Example 7.

By changing the weight gain of the minitablets when applying the inner coat, the release profile can be shifted up or down

The release profile may be changed using mixtures of mintablet fractions having different amounts of inner coating applied. The release profile may also be changed by coating with other acrylic resin such as Eudragit RL 30 D, Eudragit RS 30 D or combinations thereof, or using other types of film forming agents such as ethylcellulose or silicone polymers.

The above mentioned filmforming agents can also be combined with pore forming agents such as, e.g., cellulose ethers, polyoles, PEG's, etc.

Furthermore, the release profile may be changed applying an enteric coating to a fraction of coated minitablets.

### Example 9

### Preparation of a Controlled Release Composition Having a Release Kinetic Different From That of Zero Order

Matrix minitablets:

| | | | |
|---|---|---|---|
| I | Desglymidodrine | 800.0 | g |
| II | Ethyl cellulose (10 μm) | 2960.0 | g |
| III | Talc | 200.0 | g |
| IV | Magnesium stearate | 40.0 | g |
| V | Purified water | 800.0 | g |

I+II are admixed in a Fielder intensive mixer at an appropriate time and mixing intensity.

The mixture is wetted with V while mixing at an appropriate mixing intensity.

The wetted mixture is granulated at an appropriate time and mixing intensity. The drying of the wet granulate is carried out in an Aeromatic fluid bed.

The dried granulate is passed through a suitable sieve. III+IV are sieved through a 0.3 mm sieve and admixed to the sieved particulate mixture in a cube mixer for 10 min.

The thus obtained particulate mixture is compressed into tablets weighing 15 mg.

A dose of 30 mg desglymidodrine is contained in 10 minitablets.

If suitable, the release profile can be changed by using other cellulose ethers such as HPC, L-HPC, HPMC or combinations of thereof.

The principle of a matrix composition may also be used for a single unit tablet containing the total amount of desglymidodrine in one unit.

In order to further increase the retardation of the dissolution of desglymidodrine the minitablets may be coated with inner and outer coatings corresponding to the description of coating suspension 2 and 3 in Example 7. The amount of coating applied may be varied to shift the dissolution profile up or down.

### Example 10

### Preparation of a Controlled Release Composition Having Release Kinetic Different Form Zero Order

Matrix minitablets:

| | | | |
|---|---|---|---|
| I | Desglymidodrine | 800.0 | g |
| II | Ethyl cellulose (10 μm) | 2960.0 | g |
| III | Talc | 200.0 | g |
| IV | Magnesium stearate | 40.0 | g |
| V | Isopropyl alcohol | 800.0 | g |

I+II are admixed in a Fielder intensive mixer at an appropriate time and mixing intensity.

The mixture is wetted with V while mixing at an appropriate mixing intensity. The wetted mixture is granulated for an appropriate time and mixing intensity.

The drying of the wet granulate is carried out in an Aeromatic fluid bed.

The dried granulate is passed through a suitable sieve. III+IV are sieved through a 0.3 mm sieve and admixed to the sieved particulate mixture in a cube mixer for 10 min.

The thus obtained particulate mixture is compressed into tablets weighing 15 mg.

A dose of 30 mg of desglymidodrine is contained in 10 minitablets.

In order to further increase the retardation of the dissolution of desglymidodrine the minitablets may be coated according to Example 7. The amount of coating applied may be varied to shift the dissolution profile up or down.

### Example 11

### Composition Made by Employment of Double Compression

A tablet was prepared from the following ingredients:

| | | | |
|---|---|---|---|
| 1^{st} compression layer | Desglymidodrine | 1.93 | g |
| | Starch 1500 | 89.93 | g |
| | Lactose monohydrate | 180.00 | g |
| | Eudragite RS 30 D | 75.0 | g |
| | Acetyl tributylcitrate | 5.64 | g |
| 2^{nd} compression layer | Desglymidodrine | 0.4 | g |
| | Hydroxypropylmethyl- | 49.6 | g |
| | cellulose E 50 | | |

The granulate for 1^{st} compression layer is prepared in the following way: Desglymidodrine and Starch 1500 was mixed by hand. This mixture and lactose monohydrate is mixed in a Moulinex food processor for 30 sec.

The granulating fluid comprising Eudragit RS 30 D and acetyl tributylcitrate is mixed by stirring for 5 min

The granulating fluid was applied to the powder mixture while mixing in the Moulinex food processor. The time for applying the granulating fluid is 45 sec.

Wet massing time for the moist powder mixture is 30 sec.

The moist granulate is tray dried and the dried granulate is passed through a 1000 μm screen.

The granulate for 2^{nd} compression layer is prepared in the following way: Desglymidodrine and hydroxypropylmethyl cellulose E 50 are mixed by hand and passed through a 500 μm screen.

A double compression tablet is prepared in the following way: A shallow concave round punch of 11 mm in diameter is used to compress the tablet. 250 mg granulate for 1^{st} compression layer is weighed into the die and compressed gently to a loose compact. 200 mg granulate for 2^{nd} compression layer is weighed on top the loose compact. The loose compact and the granulate for 2^{nd} compression layer is compressed with a force of approx. 17 kN to form a coherent tablet.

After compression a release controlling film, a film containing desglymidodrine and a blank film is applied to the tablets.

The following films are applied to each tablet:

| | | | |
|---|---|---|---|
| Release | Purified water | 42.23 | mg |
| controlling film: | Hydroxypropylmethyl cellulose E 5 | 0.51 | mg |
| | Talc, pond. + Magnesium stearate | 1.04 | mg |
| | (9 + 1) | | |
| | Eudragite NE 30 D | 33.30 | mg |
| Film containing | Purified water | 72.61 | mg |
| desglymidodrine: | Hydroxypropylmethyl cellulose E 5 | 4.06 | mg |
| | Desglymidodrine | 0.81 | mg |
| | Propylene glycol | 0.81 | mg |
| Blank film: | Purified water | 32.67 | mg |
| | Hydroxypropylmethyl cellulose E 5 | 1.80 | mg |
| | Talc, pond | 1.27 | mg |
| | Propylene glycol | 0.36 | mg |

### Example 12

Preparation of a Controlled Release Composition Made in the Form of Capsules Containing Multiple Units

A desglymldodrine controlled release product is prepared by manufacturing one type of pellet, which afterwards is coated with different types of film coatings. The capsule ends up with 3 different types of pellets (one non-coated pellet, one controlled release (CR)-coated pellet and one enteric coated (EC)-pellet).

Pellet Preparation

The pellet is prepared by the use of an extrusion/spheronization technique. The ingredients are listed in Scheme 12-1.

Scheme 12-1:

| Ingredients | Amount (g) pr. Batchsize |
|---|---|
| Microcrystalline | 2146.0 |
| cellulose | |
| Lactose monohydrate | 1213.7 |
| Carmellose sodium | 70.0 |
| Desglymidodrine | 70.3 |
| Purified water | 2000.0 |

The ingredients are mixed and wetted in a Fielder high shear mixer in which the water is applied by a nozzle.

The wetted mass is extruded in a Nica E 140 extruder with a screen size of 600 μm (those pellets which are being used for non coated pellets and for CR-coating) or 800 μm (those pellets used for EC-coating). The extrudate is spheronized in a laboratory unit for 5 min. The pellets are dried in a laboratory scale fluid bed for approx 75 min at 50° C.

The dried pellets used for non coated pellets and for CR-coating are passed through a screen of 700 μm and the dried pellets used for EC-coating are fractionated wth a lower screen of 500 μm and a upper screen of 1000 μm.

Step 1 Pellets (Non Coated Pellets)

One batch of these pellets is not coated, as it is used as an immediate release unit. The pellets are a part of the content in the capsule.

Step 2 Pellets (CR-Coated Pellets)

One batch of these pellets is coated with an inner coat and an outer coat in a fluid bed (GPCG3) with a 0.8 mm spray nozzle and a spray pressure of 2.5 bar. The composition for the coating is shown in Scheme 12-2.

Scheme 12-2:

| | |
|---|---|
| | Amount (g) pr. |
| Ingredients | batchsize |

Inner coat (batchsize 2000 g)

| | |
|---|---|
| Hypromellose (viscosity | 13.1 |
| 5 cps) | |
| Purified water | 1094.0 |
| Magnesium stearate | 2.7 |
| Talc | 26.2 |
| Polyacrylate dispersion | 864.0 |
| 30% | |
| (Eudragit NE30D) | |

Outer coat (batchsize 1000 g)

| | |
|---|---|
| Hypromellose (viscosity | 40.0 |
| 5 cps) | |
| Purified water | 920.0 |
| Talc | 40.0 |

In the coating process the following amount of inner and outer coats are applied. The amount of dry matter applied calculated in percentage of the core weight also appears from below.

Inner coat: 1788.1 g per 3000.0 g pellets (dry matter: 9% of the core weight)

Outer coat: 375.0 g per 3000.0 g pellets (dry matter: 1% of the core weight)

Throughout the coating process the bed temperature is maintained substantially in the interval from 20-25° C. by adjustment of the liquid flow rate or the inlet temperature. The inlet air temperature is kept at approximately 32° C. After application of the coatings the coated pellets are cured at a bed temperature of approximately 70° C. for 30 min. Then the pellets are passed through a screen 1.0 mm. Oversized material is discarded.

Step 3 Pellets (EC Pellets)

One batch of these pellets is coated with an EC-coat in a fluid bed (Würster technique) with a 0.8 mm spray nozzle and a spray pressure of 2.5 bar. The composition for the coating is shown in Scheme 12-3.

Scheme 12-3:

| Ingredients | Amount (g) pr. batchsize |
|---|---|
| Isopropyl alcohol | 3852.0 |
| Talc | 100.0 |
| Acetyltributyl citrate | 99.2 |
| Methacrylic acid - methyl | 3948.8 |
| methacrylate copolymer (1:2) | |
| (Eudragit S12.5) | |

In the coating process the following amount of the coat is applied. The amount of dry matter applied calculated in percentage of the core weight also appears from below.

15,517.2 g per 3000.0 g pellets (dry matter: 45% of the core weight)

Throughout the coating process the bed temperature is maintained substantially in the interval from 30-38° C. by adjustment of the liquid flow rate or the inlet temperature. The inlet air temperature is kept at approximately 49° C. After the application of the coating the pellets are passed through a screen 1.3 mm. Oversized material is discarded.

Capsule Filling

The 3 different pellets (steps 1, 2 and 3) are filled into capsules by hand. The amount of pellets per capsule is shown in Scheme 12-4.

Scheme 12-4:

| Unit | Amount (mg) per capsule |
|---|---|
| Capsule | approx. 76.3 |
| Pellets step 1 | Approx. 50.4 corresp. to 1.00 mg desglymidodrine |
| Pellets step 2 | Approx. 110.6 corresp. to 2.0 mg desglymidodrine |
| Pellets step 3 | Approx. 72.7 corresp. to 1.0 mg desglymidodrine |
| Total weight | Approx. 310 corresp. to 4.0 mg desglymidodrine |
| of capsule | |

### Example 13

### Composition Made by Employment of Mixing a Matrix Granulate and a Slow Release Granulate

The composition of the granulates are equal to the granulates described in Example 11.

250 mg of granulate called 1^{st} compression layer (slow release granulate) is mixed with 200 mg of granulate called 2^{nd} compression layer (matrix granulate).

A shallow concave round punch 11 mm in diameter is used to compress the tablet. The granulate mixture is placed in the die and the granulate is compressed with a compression force of approx. 17 kN to form a coherent tablet.

After compression a release controlling film, a film containing desglymidodrine and a blank film were applied to the tablets.

The film compositions and the applied amounts are equal to the compositions and amounts applied in Example 11.

### Example 14

### Preparation of Desglymidodrine Plain Tablets

The present example illustrates the preparation of plain tablets containing desglymidodrine as the active drug substance.

Composition:

| | | |
|---|---|---|
| I | Colloidal anhydrous silica | 1.6 g |
| II | Desglymidodrine hydrochloride | 1.6 g |
| III | Microcrystalline cellulose | 14.3 g |
| IV | Maize starch | 28.84 g |
| V | Granulation liquid | 27.5 g |
| VI | Talc | 2.3% |
| VII | Magnesium stearate | 1.6% |
| V | Granulation liquid: | |
| | Maize starch | 18.3 g |
| | Purified water | 122 g |

II, III and IV were sieved through a 0.3 mm sieve. II was mixed by hand with equal parts of III, I+IV were mixed in the same way.

Hereafter I, II, III and IV were mixed in 5 minutes in a cube mixer. The mixture was wetted with V while mixing at an appropriate mixing intensity.

The moist granulate was passed through a 1000 μm sieve and tray dried at 52° C. for 3 hours.

The dried granulate was passed through a 1000 μm sieve. VI+VII was passed through a 300 μm sieve and mixed by hand with equal parts of the granulate. This mixture was admixed in a cube mixer for 5 min.

The obtained particulate mixture was compressed into tablets weighting 130 mg using a punch of 6 mm in diameter.

The tablets were subject to stability testning. For comparison purposes figures for stability testing of a desglymidodrine solution are included. The following initial results were obtained:

Stability of desglymidodrinein solution

Based on comparison of peak areas

| Media | O days (peak area) | 7 days (peak area) |
|---|---|---|
| Water | 1582140 | 1601999 |
| 0.1N HCl | 1548689 | 1516212 |
| Buffer pH 7.4 | 1341613 | 1289393 |
| Media | 0 days (peak area) | Autoclave 120° C. |
| Water | 100 | 100 |
| 0.1N HCl | 99 | 101 |
| Buffer pH 7.4 | 102 | 102 |

Stability of desglymidodrine

Based on a calibration curve of desglymidodrine

| Condition | 0 days | 14 days | 21 days |
|---|---|---|---|
| | Glass | | |
| 25° C./60% RH | 100 | 103 | 100 |
| 25° C./60% RH 1.2mioluxh | — | 103 | — |
| 40° C./75% RH | — | 102 | 100 |
| | Open petri dish | | |
| 25° C./60% RH | 100 | 102 | 100 |
| 40° C./75% RH | — | 102 | 97 |

Stability of desglymidodrine plain tablets 4 mg

Based on calculated curved of desglymidodrine

| | Glass | | |
|---|---|---|---|
| Condition | 0 days | 14 days | 21 days |
| 25° C./60% RH | 4.0 | 4.0 | 4.0 |
| 25° C./60% RH 1.2mioluxh | — | 4.0 | — |
| 40° C./75% RH | — | 4.0 | 3.9 |

Based on calculated curved of desglymidodrine

| | Open petridish | | |
|---|---|---|---|
| Condition | 0 days | 14 days | 21 days |
| 25° C./60% RH | 4.0 | 4.1 | 4.0 |
| 40° C./75% RH | — | 4.0 | 3.9 |

The results shown above clearly indicates that desglymidodrine in the specific tablet composition is stable for at least 21 days. The experiments have also be conducted under stressed temperature conditions (40° C.), and it is contemplated that the shelf-live of a composition according to the invention, i.e. the time period from the manufacture of the composition and until the content of desglymidodrine in the composition has changed more than ±10% w/w, at room temperature is at least 6 months such as, e.g. at least 1 year, at least 1.5 years, at least 2 years, at least 2, 5 years, 3 years, 4 years or 5 years.

### Example 15

### Powder Preparation e.g. for Use in a Needle-Free Device

Composition:

| | |
|---|---|
| Desglymidodrine | 1000.0 g |

The particle size distributions for the ingredients should be appropriate for deposition of the composition in an appropriate layer of the skin e.g. 0.5 μm to 10 μm.

The powder is filled into a drug casette, each containing 4 mg desglymidodrine.

Suspending agents such as glucose, lactose, celluloses, starches (maize-, rice-, potato-), calcium phosphate or mixtures of these may be used.

### Example 16

### Liquid Composition for Use in a Needle-Free Device

Composition:

| | | |
|---|---|---|
| I | Desglymidodrine | 40.0 g |
| II | Sodium edetate | 0.5 g |
| | Disodium hydrogen phosphate dihydrate | 2.0 g |
| | Sodium dihydrogen phosphate dihydrate | 2.0 g |
| III | Water for injection | 900.0 g |
| IV | Water for injection | ad 1000.0 g |

I is dissolved in III upon continuous stirring. The remaining solid ingredients (II) are added to the solution one by one during continuous stirring. Following complete dissolution of the solids, purified water is added to a total weight of 1000.0 g.

The formulation is filtrated (0.22 μm) and is filled into glass devices with a piston (e.g. teflon) and a stopper (e.g. rubber (natural or synthetic substances)).

Tonicity agents may be dextrose, glycerol, sorbitol, mannitol, potassium nitrate and sodium sulphate decahydrate or mixtures thereof.

pH may be adjusted to the appropriate value by use of additional buffer salts such as citric acid, sodium citrate, and potassium dihydrogen phosphate or mixtures of these.

### Example 17

### Liquid Compositions for Pulmonal Delivery

### Example 17.1

### Pressurised Metered-Dose Preparation for Inhalation

Composition:

| | | |
|---|---|---|
| I | Desglymidodrine | 40.0 g |
| II | Norflurane | ad 1000.0 ml |

I is dissolved or suspended in liquid II at low temperature during continuous agitation. For suspensions, the particle size distribution should be appropriate for deposition of the composition in the lung, e.g. 0.5 μm to 10 μm. The product is filled into suitable pressurised multi-dose containers delivering e.g. 100 μl pr. dose.

Other propellants such as dichlorodifluoromethane, dichlorotetrafluoroethane and trichlorofluoromethane or mixtures of these may be used.

Glidants such as oleic acid and derivatives and isopropyl myristate or mixtures of these may be used to reduce friction during administration.

### Example 17.2

### Liquid for Nebulisation

Composition:

| | | |
|---|---|---|
| I | Desglymidodrine | 2.0 g |
| II | Sodium edetate | 0.5 g |
| | Disodium hydrogen phosphate dihydrate | 2.0 g |
| | Sodium dihydrogen phosphate dihydrate | 2.0 g |
| III | Purified water | 900.0 g |
| IV | Purified water | ad 1000.0 g |

I is dissolved in III upon continuous stirring. The remaining solid ingredients (II) are added to the solution one by one during continuous stirring. Following complete dissolution of the solids, purified water is added to a total weight of 1000.0 g. The composition is filled into 2 ml ampoules or other suitable unit-dose containers.

Other excipients may also be added such as polyethylene glycol, alcohol, glycofurol, phospholipids, poloxamer, polyoxyethylene castor oil derivatives, polysorbates, propylene glycol and cyclodextrins or combinations of these. Tonicity agents may be e.g dextrose, glycerol, sorbitol, mannitol, sodium chloride, potassium nitrate and sodium sulphate decahydrate or mixtures thereof.

pH may be adjusted to the appropriate value by use of additional buffer salts such as citric acid, sodium citrate, and potassium dihydrogen phosphate or mixtures of these. Sufficient microbiological preservation may be achieved by addition of benzalconium chloride or parabenes. Suitable flavours can be added to the composition and use of sweeteners such as saccharin, acesulfame, aspartame, cyclamate salts or mixtures of these can further adjust the taste.

### Example 18

### Powder Composition for Pulmonal Delivery

Composition:

| | | |
|---|---|---|
| I | Desglymidodrine | 400.0 g |
| II | Glucose | 500.0 g |

The particle size distributions for the ingredients should be appropriate for deposition of the composition in the lung, e.g. 0.5 μm to 10 μm. I and II are carefully mixed and sieved. The powder is filled into capsules or other suitable unitdose containers, each containing 10 mg of the composition.

Other suspending agents such as lactose, celluloses, starches (maize-, rice-, potato-) calcium phosphate or mixtures of these may be used.

### Example 19

### Nasal Compositions

General Description of a Nasal Composition

A nasal composition of desglymidodrine is buffered, and tonicity adjusted and it can be delivered from a device, which may or may not require the presence of antimicrobial agents in the composition. The amount of desglymidodrine reaching the systemic circulation may be increased by addition of absorption enhancer(s) to the composition.

The Variability of a Nasal Composition

The total amount of absorption enhancers included in the composition will, typically, vary between 0.01% and 10%. However, some absorption enhancers may also serve as vehicles and thereby totally replace the content of water in the composition. One can include one, two or several absorption enhancing agents in the composition. The final nasal composition may be a homogenous liquid, a suspension, an emulsion, a gel or a powder. The dose administered intranasally may be adjusted by choice of the volume of the formulation, ranging from 10 μl to 250 μl per nostril or of the mass of the composition, ranging from 5 mg to 50 mg.

### Specific Examples of Nasal Compositions

### Example 19.1

### Nasal Composition Without Absorption Enhancer

| Composition: | | | Function of ingr. |
|---|---|---|---|
| I | Desglymidodrine | 40.0 g | Active ingr. |
| II | Sodium edetate | 0.5 g | Stabiliser |
| | Disodium hydrogen phosphate | 2.0 g | Buffer component |
| | dihydrate | | |
| | Sodium dihydrogen phosphate | 2.0 g | Buffer component |
| | dihydrate | | |
| III | Purified water | 900.0 g | Solvent |
| IV | Purified water | ad to 1000.0 g | Solvent |

I is dissolved in III upon continuos stirring. The remaining solid ingredients (II) are added to the solution one by one during continuos stirring. Following complete dissolution of the solids, purified water is added to a total weight of 1000.0 g.

The composition is filled into appropriate nasal spray devices delivering e.g. 100 μl pr.dose

pH may be adjusted to the appropriate value by use of additonal buffer salts such as citric acid, sodium citrate and potassium dihydrogen phosphate or mixtures of these. Sufficient microbiological preservation may be achieved by addition of benzalconium chloride, sorbic acid or parabenes such as methylparaben, ethylperaben, propylparaben and butylparaben or mixtures of these.

### Example 19.2

### Nasal Composition With Absorption Enhancer

| Composition: | | | Function of ingr. |
|---|---|---|---|
| I | Desglymidodrine | 40.0 g | Active ingr. |
| II | Sodium glycocholate | 5.0 g | Abs. enhancer |
| III | Sodium edetate | 0.5 g | Stabiliser |
| | Disodium hydrogen phosphate | 2.0 g | Buffer component |
| | dihydrate | | |
| | Sodium dihydrogen phosphate | 2.0 g | Buffer component |
| | dihydrate | | |
| IV | Purified water | 900.0 g | Solvent |
| V | Purified water | to 1000.0 g | Solvent |

II is dissolved in IV, and I is added upon continuous stirring, The remaining solid ingredients (III) are added to the solution one by one during continuos stirring. Following complete dissolution of the solids, purified water is added to a total weight of 1000.0 g.

The composition is filled into appropriate nasal spray devices delivering e.g. 100 μl pr.dose.

One, two or several of the absorption enhancers sodium cholate, sodium deoxycholate, sodium taurocholate, sodium taurodeoxycholate, sodium glycodeoxycholate, α-cyclodextrin, β-cyclodexrin, γ-clodextrin, methyl cyclodextrn, hydroxypropyl-β-cyclodextrin, dimethyl-β-cyclodextrin, sodium taurodihydrofusidate, phosphatidylcholines, chitin, chitosan, hyaluronic acid, polyetylene glycols, starch microspheres and dextran microspheres may be included in the composition.

pH may be adjusted to the appropriate value by use of additional buffer salts such as citric add, sodium citrate and potassium dihydrogen phosphate or mixtures of these. Sufficient microbiological preservation may be achieved by addition of benzalconium chloride, sorbic acid or parabenes such as methylparaben, ethylparaben, propylparaben and butylparaben or mixtures of these.

General Description of a Composition Containing Liposomes

Liposomes, or lipid vesicles, are spherical self-dosed structures composed of concentric bilayers that entrap part of the solvent or active drug substance in the central core or in the bilayer depending on the hydrophilicity of the active drug substance. Liposomes are predominantly made of natural amphiphilic lipids, e.g. phospolipids. Liposomes are likely to enhance the penetration of the active ingredient into the mucosa such as, e.g. the nasal, buccal, oral, rectal or vaginal mucosa.

Description of the Variability of a Liposomal Composition

The liposomes can be based on liposome forming lipids and liposome stabilising or destabilising lipids. The total amount of lipids in the composition can vary between 20-80% w/w. The ratio between liposome forming lipids and stabilising/destabilising lipids can be between 1:1 to 40:1 (on a molar basis) or the composition can contain liposome orming lipids alone. One can include one, two or several liposome forming lipids in the composition.

### Example 19.3

### Nasal Composition With Liposomes

| Composition: | | | Function of ingr. |
|---|---|---|---|
| I | Desglymidodrine | 40.0 g | Active ingr. |
| | Disodium hydrogen phosphate | 2.0 g | Buffer component |
| | dihydrate | | |
| | Sodium dihydrogen phosphate | 2.0 g | Buffer component |
| | dihydrate | | |
| II | Purified water | 900.0 g | Solvent |
| III | Purified water | to 1000.0 g | Solvent |
| IV | DSPC | 300.0 g | Liposome forming |
| | CH | 84.0 g | Liposome |
| | | | stabiliser/ |
| | | | destabiliser |

I is dissolved in II upon continuos stirring. Following complete dissolution of the solids, purfied water is added to a total weight of 1000.0 g.

The dry mix of DSPC:CH (7 mol DSPC:2 mol CH) (IV) is dispersed in water, and dehydrated. The liquid composition containing desglymidodrine is poured into the dehydrated DSPC:CH during vigorous stirring to rehydrate the mixture of DSPC:CH.

pH may be adjusted to the appropriate value by use of additional buffer salts such as citric acid, sodium citrate and potassium dihydrogen phosphate or mixtures of these. Sodium edetate may be added to the composition as stabiliser. Sufficient microbiological preservation may be achieved by addition of sorbic acid or parabenes such as methylparaben, ethylparaben, propylparaben and butylparaben. Propylene glycol (10%) can be added to potentiate the antimicrobial activity of the parabens in the presence of some amphiphilics.

The composition is filled into appropriate nasal spray devices delivering the desired dose. The volume of the dose can be between 10-250 μl, preferably 100 μl. The dose can also be administrered by application of two puffs, one in each nostril.

Other vesicle forming lipids can also be used instead of DSPC in the lipid bilayer. These amphiphilic lipids may be cationic, anionic or neutral, such as DPPC, DLPC, DOPC, DSEPC, dialkyl (C₈-C₂₀) sulfosuccinate or fatty alcohol ethoxylate (with alkyl chain length of C₈-C₂₀ and 4 to 6 ethoxy groups). The molecular amount of lipids compared to active compound can be optimised with different liposome building lipids.

CH can be replaced by cholesterol derivatives or any other stabiliser/destabiliser such as alkyl (C₈-C₂₀) phosphate, alkyl (C₈-C₂₀) sulfate, alkyl (C₈-C₂₀) ethersulfate, alkyl (C₈-C₂₀) ether carboxylate. Further stabilisers/destabilisers can be employed such as stearoyl lysophosphatidyl choline, lysophosphatidylcholine, palmitoyl lysophosphatidyl choline and didecanoyl phosphatidyl choline.

### Example 20

### Buccal Compositions

General Description of a Buccal Composition

A buccal composition of desglymidodrine is buffered and tonicity adjusted. It can be delivered from a device, which may or may not require the presence of antimicrobial agents in the formulation. The amount of desglymidodrine reaching the systemic circulation may be increased by addition of absorption enhancer(s) to the composition.

The Variability of the Buccal Composition

The total amount of absorption enhancers included in the composition will, typically, vary between 0.01% and 10%. However, some absorption enhancers may also serve as vehicles and thereby totally replace the content of water in the formulation. One can include one, two or several absorption enhancing agents in the formulation. The final buccal formulation may be a homogenous liquid, a suspension, an emulsion, a gel or a powder. The dose administered buccally may be adjusted by choice of the volume of the formulation, ranging from 10 μl to 500 μl or the mass of the composition, ranging from 5 mg to 100 mg.

### Example 20.1

### Buccal Composition With Liposomes

| Composition: | | | Function of ingr. |
|---|---|---|---|
| I | Desglymidodrine | 40.0 g | Active ingr. |
| | Disodium hydrogen phosphate | 2.0 g | Buffer component |
| | dihydrate | | |
| | Sodium dihydrogen phosphate | 2.0 g | Buffer component |
| | dihydrate | | |
| II | Purified water | 900.0 g | Solvent |
| III | Purified water | to 1000.0 g | Solvent |
| IV | DSPC | 300.0 g | Liposome forming |
| | CH | 84.0 g | Liposome stabiliser/ |
| | | | destabiliser |

I is dissolved in II upon continues stirring. Following complete dissolution of the solids, purified water is added to a total weight of 1000.0 g.

The dry mix of DSPC:CH (7 mol DSPC:2 mol CH) (IV) is dispersed in water, and dehydrated. The liquid composition containing desglymidodrine is poured into the dehydrated DSPC:CH during vigorous stirring to rehydrate the mixture of DSPC:CH.

pH may be adjusted to the appropriate value by use of additional buffer salts such as citric acid, sodium citrate and potassium dihydrogen phosphate or mixtures of these. Sodium edetate may be added to the composition as stabiliser. Sufficient microbiological preservation may be achieved by addition of sorbic acid or parabenes such as methylparaben, ethylparaben, propylparaben and butylparaben. Propylene glycol (10%) can be added to potentiate the antimicrobial activty of the parabens in the presence of some amphiphilics.

The composition is filled into appropriate buccal spray devises delivering the desired dose. The volume of the dose can be between 10-500 μl or the dose can be administrered by application of multiple puffs.

Other vesicle forming lipids can also be used instead of DSPC in the lipid bilayer. These amphiphilic lipids may be cationic, anionic or neutral, such as DPPC, DLPC, DOPC, DSEPC, dialkyl (C₈-C₂₀) sulfosuocinate or fatty alcohol ethoxylate (with alkyl chain length of C₈-C₂₀ and 4 to 6 ethoxy groups). The molecular amount of lipids compared to active compound can be optimised with different liposome building lipids.

CH can be replaced by cholesterol derivatives or another stabiliser/destabiliser such as alkyl (C₈-₂₀) phosphate, alkyl (C₈-C₂₀) sulfate, alkyl (C₈-C₂₀) ethersulfate, alkyl (C₈-C₂₀) ether carboxylate.

### List of Abbreviations

| | |
|---|---|
| CH | cholesterol |
| DSPC | distearoyl glycero phosphatidyl choline |
| DSEPC | distearoyl glycero ethyl phosphatidyl choline |
| DPPC | dipalmitoyl phosphatidyl choline |
| DLPC | dilauroyl phosphatidyl choline |
| DOPC | dioleoyl phosphatidyl choline |
| PC | phosphatidyl choline |

### Example 20.2

### Buccal Composition Without Absorption Enhancer

| Composition: | | | Function of ingr. |
|---|---|---|---|
| I | Desglymidodrine | 40.0 g | Active ingr. |
| II | Sodium edetate | 0.5 g | Stabiliser |
| | Disodium hydrogen phosphate | 2.0 g | Buffer component |
| | dihydrate | | |
| | Sodium dihydrogen phosphate | 2.0 g | Buffer component |
| | dihydrate | | |
| III | Purified water | 900.0 g | Solvent |
| IV | Purified water | to 1000.0 g | Solvent |

I is dissolved in III upon continuos stirring. The remaining solid ingredients (II) are added to the solution one by one during continuos stirring. Following complete dissolution of the solids, purified water is added to a total weight of 1000.0 g.

The composition is filled into appropriate buccal spray devices delivering e.g. 100 μl pr.dose.

pH may be adjusted to the appropriate value by use of additional buffer salts such as citric acid, sodium citrate and potassium dihydrogen phosphate or mixtures of these. Sufficient microbiological preservation may be achieved by addition of benzalconium chloride, sorbic acid or parabenes such as methylparaben, ethylparaben, propylparaben and butylparaben or mixtures of these.

### Example 20.3

### Buccal Composition With Absorption Enhancer

| Composition: | | | Function of ingr. |
|---|---|---|---|
| I | Desglymidodrine | 40.0 g | Active ingr. |
| II | Sodium glycocholate | 5.0 g | Abs. enhancer |
| III | Sodium edetate | 0.5 g | Stabiliser |
| | Disodium hydrogen phosphate | 2.0 g | Buffer component |
| | dihydrate | | |
| | Sodium dihydrogen phosphate | 2.0 g | Buffer component |
| | dihydrate | | |
| IV | Purified water | 900.0 g | Solvent |
| V | Purified water | to 1000.0 g | Solvent |

II is dissolved in IV, and I is added upon continuous stirring. The remaining solid ingredients (III) are added to the solution one by one during continues stirring. Following complete dissolution of the solids, purified water is added to a total weight of 1000.0 g.

The composition is filled into appropriate buccal spray devices delivering e.g, 100 μl pr.dose.

One, two or several of the absorption enhancers sodium cholate, sodium deoxycholate, sodium taurocholate, sodium taurodeoxycholate, sodium glycodeoxycholate, α-cyclodextrin, β-cyclodextrin, γ-cyclodextrin, methyl cyclodextrin, hydroxypropyl-β-cyclodextrin, dimethyl-β-cyclodextrin, sodium taurodihydrofusidate, phosphatidylcholines, chitin, chitosan, hyaluronic acid, polyetylene glycols, starch microspheres and dextran microspheres may be included in the composition.

pH may be adjusted to the appropriate value by use of additional buffer salts such as citric acid, sodium citrate and potassium dihydrogen phosphate or mixtures of these. Sufficient microbiological preservation may be achieved by addition of benzalconium chloride, sorbic acid or parabenes such as methylparaben, ethylparaben, propylparaben and butylparaben or mixtures of these.

### Example 21

### Desglymidodrine Sublingual Tablets

General Description

Sublingual tablets are designed to give a fast disintegration in the mouth by the amount of salvia normally available in that region. The disintegration time is therefore very short or short and there may be additives, which promote salvia secreting in the composition.

A fast disintegration together with a high solubility of the drug substance give the possibility of a fast absorption through the mucous membrane of the mouth, especially under the tongue where the blood vessels run close to the surface.

Sublingual absorbed drugs avoid more or less the first pass liver metabolism.

Description of Variability in Sublingual Compositions

Sublingual tablets may be prepared by mould technique, by direct compression or by conventional wet granulation or dry granulation (e.g. roller compaction) of the ingredients. As mentioned, taste improving substances may be added to promote salvia secretion. This may include artificial sweeteners (cyclamate, saccharin sodium, aspartame, etc.), natural sweeteners (saccharose, sorbitol, xylitol, etc.), week organic acids (citric-, acetic-, ascorbic acid, etc.) natural or artificial flavours (strawberry, black currant, pineapple, apple, orange, lemon, etc.) in the below mentioned compositions.

### Example 21.1

| Composition: | | | | Function |
|---|---|---|---|---|
| I | Desglymidodrine | 2.0 g | 4.0 g | Active ingr. |
| II | Purified water | | | Solvent |
| | Ethanol 96% equal parts | q.s. | q.s. | Moist. agent |
| III | Lactose | 53 g | 51 g | Filler |
| | Mannitol | 45 g | 45 g | Filler |

The amounts of lactose and mannitol may be varied from 25 to about 100 g. Dose 100 mg (50-200 mg) corresponds to 2.0 or 4.0 mg desglymidodrine of the two compositions, respectively.

Disintegration time is extremely short (2-5 sec).

II is prepared, and I is dissolved in II. The solution is incorporated in III to a homogeneous mixture is achieved. More II may be added.

The moistured mass is spread on a suitable plate equipped with wholes into which the mass is pressed and which have a depth that gives the wanted dose of drug substance. The wet mass is pressed out of the wholes and allowed to dry before further handling. (Sublingual tablet preparation by mould-technique).

II may be replaced by mixtures of different volatile organic solvents and may have a water content of 0-90%. III may be replaced by different mixtures of soluble pharmaceutically acceptable excipients as sorbitol, mannitol, xylitol, maltodextrin, lycasin, lactitol etc.

### Example 21.2

| Composition: | | | | Function |
|---|---|---|---|---|
| I | Desglymidodrine | 2.0 g | 4.0 g | active ingr. |
| II | Tablettose | 55.0 g | 55.0 g | filler |
| III | Cellulose, microcryst. | 10.0 g | 10.0 g | filler/binder |
| | Type 102 | | | |
| IV | Agar sieve 180 | 5.0 g | 5.0 g | disintegrant |
| V | Magnesium stearate | 0.5 g | 0.5 g | glidant/lubricant |
| | Talc | 4.5 g | 4.5 g | glidant/lubricant |

I is mixed with II and further with III, IV and V and compressed into tablets.

The amount of the filler, tablettose, may vary from 25 to 75 g, the filler/binder (cellulose) may vary from 5 to 25 g, the disintegrant from 1 to 15 g, magnesium stearate from 0.1 to 2.5 g and talc from 1.0 to 10 g.

Mass weight of 77 mg corresponds to a dose of 2.0 mg or mass weight of 79 mg corresponds to a dose of 4.0 mg desglymidodrine in the two compositions, respectively.

Disintegration time is short (30 sec-4 min).

Tablettose may be replaced by other qualities of lactose with good flowability. Agar may be replaced by other disintergrants as croscarmellose sodium/calcium or the like, crosspovidone, starch glycolate, alginates or other disintegrants. Magnesium stearate may be replaced by other glidants as different types of silica colloidal hydrous or anhydrous, Ca-stearate, stearic acid, sodium stearylfumarate, cotton-seed oil, hydrogenated vegetable oils or other suitable lipid substances as e.g. Myvatex.

### Example 21.3

| Composition: | | | | Function |
|---|---|---|---|---|
| I | Desglymidodrine | 2.0 g | 4.0 g | active ingr. |
| II | Maize starch | 9.5 g | 7.0 g | filler |
| | Lactose | 66 g | 66 g | filler |
| III | Povidone 30 | 2.5 g | 2.5 g | binder |
| IV | Purified water | q.s. | q.s | solvent |
| V | Mannitol | 36 g | 36 g | filler/taste |
| VI | Magnesum stearate | 1.5 g | 1.5 g | lubricant |

I is mixed with II and granulated with III dissolved in IV. More IV may be added. After drying, V and VI are added and tablets are compressed.

The amount of maize starch may vary from 5 to 15 g, lactose from 30 to 100 g, mannitol from 10 to 80 g, and magnesium stearate from 0.5 to 5 g.

Disintegration time is about 5 min.

Maize starch may be replaced by other suitable starches such as rice- or potato starch. Lactose may be replaced by maltodextrine, dextrin etc. Povidone 30 may be replaced by povidone VA 64 or 90 or gelatine or pregelatinzed starch or different types of cellulose (methylcellulose, hydroxypropyl cellulose etc.). Mannitol may be replaced by sorbitol, xylitol, maltitol, maltodextrin, lactitol, etc. Magnesum stearate may be replaced by other glidants as different types of colloidal silica hydrous or anhydrous, Ca-stearate, stearic acid, sodium stearylfumarate, cotton-seed oil, hydrogenated vegetable oils or other suitable lipid substances as e.g. Myvatex.

### Example 22

### Desglymidodrine Melt Tablets

General Description of Melt Tablets

Melt-tablets are also referred to as fast/rapidly-disintegrating tablets, dispersing tablets and dissolving tablets. In this example, the term “melt-tablet” is applied.

Melt-tablets are a tablet dosage form for oral administration, one that disintegrates instantaneously and releasing the drug, which dissolves or disperse rapidly in saliva and afterwards swallowed without the need for water. The drug substance is absorbed via the gastrointestinal tract.

Less frequently melt-tablets are designed in a way so the drug substance are to be absorbed through the buccal mucosa. In that case the bioavailability of the drug from the melt-tablet may be even greater than observed for standard dosage forms. Further more side-effects may be reduced if they are caused by first-pass metabolites.

A Description of the Variability of the Melt-Tablets

Techniques currently applied for formulate melt-tablets are namely freeze-drying (lyophilisation), spray drying, tablet moulding and direct compression. In this example only melt-tablets prepared by lyophilisation are described.

To ensure melt-tablets fast-disintegrating and dissolving attribute water must quickly progress into the tablet matrix to cause rapid disintegration and instantaneous dissolution of the tablet. Maximising the porous structure of the tablet matix and incorporating appropriate disintegrating agent and/or highly water-soluble excipients in the tablet formulation are the basic approaches applied in the current melt-tablet technologies.

In the formulation the total amount of disintegrating agents and/or highly water-soluble excipients can vary between 5-80% (w/v) and the total amount of binding agents can vary between 0.05-5% (w/v). Water is used to ensure the porous structure. Sufficient microbiological preservatives (benzoic acid, methylparaben etc.) may be added to prevent microbiological growth of the aqueous solution during production. When the product has been dried, the preservative has no further function.

Taste improving agents may be added to promote salvia secretion. This may include artificial sweeteners (aspartame, cyclamate, saccharin etc.), natural sweeteners (sucrose, sorbitol, xylitol etc.) week organic acids (citric-, ascorbic acid etc.), natural or artificial flavours (strawberry, black currant, pineapple, apple, orange, lemon etc.). Colouring agents may also be added to give the melt-tablets elegance and identity.

Permeation enhancers (sodium lauryl sulphate etc.) may be added to optimise the transmucosal absorption and pH-adjusting excipients (hydrochloride, sodium hydroxide etc.) may be used to optimise the chemical stability of the drug. Collapse protecting agents (glycine etc.) to prevent the shrinkage of the tablet during lyophilisation process or during long-term storage,

Melt-Tablet Prepared by Lyophilisation

| | Composition: | | Function of ingredient |
|---|---|---|---|
| I | Desglymidodrine | 4.0 g | Active |
| II | Maltodextrin | 200 g | Water-soluble |
| III | Xanthan gum | 20 g | Binding agent |
| IV | Purified water | 776 g | Solvent |

II and III are dissolved in IV upon continues stirring and I is added to the solution during stirring. Purified water is added to a total weight of 1000.0 gram.

The solution is filled in PVC blister with a diameter of 15 mm and a depth of 6 mm, so the resulting dose of Desglymidodrine is 4.0 mg pr. tablet,

The blisters are placed on the shelves of the freeze-dryer. Samples are frozen to −45° C. at a rate of 0.5° C./min and kept at this temperature for 1.5 hour. Primarily, drying is performed by keeping the blisters for 8 hour at a pressure of 1 mbar, a shelf temperature of −10° C., and a condenser temperature of −60° C.

Reducing the pressure to 0.1 mbar, carrying out secondary drying and increasing the shelf temperature to 25° C. Secondary drying time is 6 hour. Lyophilisation is terminated by venting the drying chamber with air.

II may be replaced by lactose, mannitol, dextrose, xylitol, fructose, sucrose, maltose, sorbitol etc. or mixtures of these. As disintegrating agent croscarmellose, crospovidone etc. may be added. Other excipients may also be used instead of III. These excipients may be gelatine, tragacanth gum, agar, acacia, alginate, dextran, povidone, hydroxyethylcellulose etc. or mixtures of these.

### Example 23

### Desglymidodrine Rectal Compositions

General Description

Rectal solution is a way of drug administration, which can be used independently of the condition of the patient. Furthermore a quite quick onset of effect is seen for some compositions. A major part of the absorbed drug dose does not undergo first pass metabolism, which in some cases may be an advantage.

Description of Variability in Composition

The amount of rectal solution to give one dose is kept on the small amount of 1-4 ml such as, e.g., 2.0 ml. This is to avoid any emptying reflex from the rectum mucosa after applying the solution in the rectum. The solution will be dispensed in a suitable container as a single-dose syringe or sealed plastic tube. Both equipped with a suitable tip.

The following examples have been chosen to ilustrate formulation of a rectal solution. 24.1: A solution with high content of pharmaceutical acceptable organic solvent to promote absorption, 24.2: A simple aqueous isotonic solution, 24.3: A simple slightly viscous aqueous isotonic, and finally 24.4: An aqueous pH-adjusted isotonic solution.

### Example 23.1

| Composition: | | | | | Function of ingredient |
|---|---|---|---|---|---|
| Desglymidodrine | 1.00 | g | 2.00 | g | Active ingr. |
| Glycerol 85% | 500 | g | 500 | g | Solvent/abs. Enhancer |
| Purified water | 1000 | ml | 1000 | ml | Solvent |

One dose of 2.0 ml=2.0 mg desglymidodrine or 2.0 ml=4.0 mg desglymidodrine. Glycerol may be exchanged with glycofurol, polythylene glycols 200 to 600, propylene glycol or similar non-irritant suitable solvent. The amount of glycerol may vary from about 200to800g.

### Example 23.2

| Composition: | | | | | Function of ingredient |
|---|---|---|---|---|---|
| Desglymidodrine | 1.00 | g | 2.00 | g | Active ingr. |
| Sodium chloride | 9.0 | g | 9.0 | g | Isotonic agent |
| Propylis paraoxibenzoate | 0.2 | g | 0.2 | g | Preservative |
| Methylis paraoxibenzoate | 0.8 | g | 0.8 | g | Preservative |
| Purified water up to | 1000 | ml | 1000 | ml | Solvent |

The mixture of paraoxibenzoates may be exchanged with other suitable preservatives.

Dose 2.0 ml=2.0 mg desglymidodrine or 2.0 ml=4.0 mg desglymidodrine

### Example 23.3

| Composition: | | | | | Function of ingredient |
|---|---|---|---|---|---|
| Desglymidodrine | 1.00 | g | 2.00 | g | Active ingr. |
| Sodium carboxymethyl- | 6.0 | g | 6.0 | g | Viscosity incr. agent |
| cellulose | | | | | |
| Propylis paraoxibenzoate | 0.2 | g | 0.2 | g | Preservative |
| Methylis paraoxibenzoate | 0.8 | g | 0.8 | g | Preservative |
| Purified water up to | 1000 | ml | 1000 | ml | Solvent |

The mixture of paraoxibenzoates may be exchanged with other suitable preservatives.

Dose 2.0 ml=2.0 mg desglymidodrine or 2.0 ml=4.0 mg desglymidodrine. The amount of viscosity increasing agent may vary from about 2 to about 8 g.

### Example 23.4

| Composition: | | | | | Function of ingredient |
|---|---|---|---|---|---|
| Desglymidodrine | 1.00 | g | 2.00 | g | Active ingr. |
| Sodium acetate | 1.0 | g | 1.0 | g | Buffer agent |
| Sodium chloride | 8.5 | g | 8.5 | g | Isotonic agent |
| Propylis paraoxibenzoate | 0.2 | g | 0.2 | g | Preservative |
| Methylis paraoxibenzoate | 0.8 | g | 0.8 | g | Preservative |
| Purified water up to | 1000 | ml | 1000 | ml | Solvent |

The mixture of parabenzoates may be exchanged by other suitable preservative. Sodium acetate may be exchanged by other suitable pH regulating substance or a buffer mixture with pH in the interval of 7.0-8.0. The amount of buffer agent may vary from about 0.5 to about 3.0 g.

Dose 2.0 ml=2.5 mg desglymidodrine or 2.0 ml=4.0 mg desglymidodrine.

### Example 24

### Desglymidodrine Oral Drops

General Description of Oral Drops

Oral drops are a dosage form for peroral administration. The formulation allows the patient to use a dose of 5-15 drops of the product and, optinally, dilute this dose in water or other better tasting liquids (e.g. orange juice) before ingestion.

Description of Variability in Composition

The amount of oral drops containing one drug dose is kept at the small amount of 5 -15 drops. The standard volume of drops is normally 10-25 ml in a bottle equipped with a suitable tip. This tip is constructed to deliver the drops with a speed suitable for counting the drops as they leave the tip, when the bottle is turned around with the button up. This aggregate has to confirm with Ph. Eur demands.

### Example 24.1

| Composition: | | | Function of ingredient |
|---|---|---|---|
| Desglymidodrine | 10.0 | g | Active ingr. |
| Ethanol 96% | 120 | g | Solvent/preservative |
| Purified water up to | 1000 | ml | Solvent |

One dose of 2.0 mg is equivalent to approximately 6 drops (have to be measured exactly), normally ingested in 20-200 ml water or other drinkable fluid.

### Example 24.2

| Composition: | | | Function of ingredient |
|---|---|---|---|
| Desglymidodrine | 10.0 | g | Active ingr. |
| Propyl paraoxibenzoate | 0.2 | g | Preservative |
| Methyl paraoxibenzoate | 0.8 | g | Preservative |
| Purified water up to | 1000 | ml | Solvent |

The mixture of paraoxibenzoates may be exchanged with other suitable preservatives.

One dose of 2.0 mg is equivalent to approximately 4 drops (have to be measured exactly), this does is normally ingested in 20-200 ml water or other drinkable fluid.

### Example 25

### Desglymidodrine Oral Solution

General Description of Oral Solution

Oral solution is for peroral administration. The oral solution is in the form of a solution of the drug substance in a suitable and well tasting vehicle.

Description of Variability in Composition

Oral solution is normally given in a volume of 2.0-15 ml measured with a suitable device, which is able to give the desired volume with the specified (Ph.Eur.) precision. Oral solution may be taste masked and may be formulated with or without sugar, furthermore viscosity-increasing substance may be added to make to make the handling and administration of the solution easier.

### Example 26.1

| Composition: | | | | | Function of ingredient |
|---|---|---|---|---|---|
| Desglymidodrine | 0.400 | g | 0.800 | g | Active ingr. |
| Sucrose | 760 | g | 760 | g | Sweetener |
| Purified water up to | 1000 | ml | 1000 | ml | Solvent |

5.0 ml equivalent to 2.0 mg or 4.0 mg Preservative may be added. 0.1N HCl may be added to adjust pH in the interval 2.5-3.5.

### Example 25.2

| Composition: | | | Function of ingredient |
|---|---|---|---|
| Desglymidodrine | 0.800 | g | Active ingr. |
| Black currant juice | 240 | g | Taste masking |
| Sorbitiol | 400 | g | Sweetener |
| Potassium sorbate | 0.14 | g | Preservative |
| Levomenthol | 0.044 | g | Taste masking |
| Ethanol 96% | 0.176 | | Solvent |
| Purified water up to | 1000 | ml | Solvent |

0.1N HCl may be added to adjust pH in the interval 2.5-3.5. The concentration of the active ingredient may be changed in the interval of 0.1 to 10 g if needed. Potassium sorbate may be exchanged with other suitable preservatives. Black currant juice may be exchanged with other fruit juices or mixtures of these. Sorbitol may be exchanged with other sweetener as mannitol, xylitol, maltodextrin, lycasin, lactitol etc. or mixtures of these. Levomenthol may be exchanged with other taste masking ingredients: natural or artificial flavours (strawberry, black currant, pineapple, apple, orange, lemon etc. or mixtures of these).

Artificial sweeteners (aspartame, cyclamate, saccharin etc) and/or week organic acids (citric-, ascorbic acid etc.) may also be added. Colours may also be added to improve the organoleptic properties.

### Example 25.3

| Composition: | | | Function of ingredient |
|---|---|---|---|
| Desglymidodrine | 0.800 | g | Active ingr. |
| Poloxamer 8000 | 6.00 | g | Solubilizer |
| Methyl parahydroxybenzoate | 1.10 | g | Preservative |
| Anis oil | 0.13 | g | Taste masking |
| Eucalyptol | 0.17 | g | Taste masking |
| Ethanol 96% | 0.176 | | Solvent |
| Hydrogenated glucose syrup | 385 | g | Sweetener |
| Purified water up to | 1000 | ml | Solvent |

0.1N HCl may be added to adjust pH in the interval 2.5-3.5. Poloxamer 8000 may be exchanged with other GRAS accepted surfactants. The composition of this example has a high content of taste masking ingredients, which explains the need for a surfactant. Dose 5.0 ml equivalents 4.0 mg desglymidodrine.

### Example 26

### Desglymidodrine Solution for Infusion or Injection

General Description of Solutions for Infusion or Injection

Solution for infusion is a ready for use solution aimed for infusion in one of the major veins. Solutions for injection may be injected i.v., s.c., i.m. or by any other suitable route. The solution is formulated as simple as possible. For stability reasons the pH may be adjusted in acidic direction and this cause the infusion time have to run over a couple of minutes. In some cases this is done by injection of a desglymldodrine solution for infusion into an already established infusion of glucose or sodium chloride.

### Example 26.1

| Composition: | | | | | Function of ingredient |
|---|---|---|---|---|---|
| Desglymidodrine | 0.400 | g | 0.800 | g | Active ingr. |
| Sodium chloride | 9.0 | g | 9.0 | g | Isotonic agent |
| Purified water up to | 1000 | ml | 1000 | ml | Solvent |

0.1N HCl at pH 2.5-3.5 may be added,

Dose: 2.0 mg in 5 ml or 4.0 mg in 5 ml. The solution may also be presented in vials or the form of a unit dose e.g. in ampoules.

### Example 27

General Description of Transdennal Drug Delivery Systems

Transdermal drug delivery systems are designed to deliver a drug substance through the skin for systemic circulation and effect. A transdermal drug delivery system can be designed to deliver a drug substancento the skin at a given rate.

Drugs reaching the systemic circulation through the skin more or less surpass the first-pass metabolism in the liver.

Description of Variability in the Transdermal Drug Delivery System

Transdermal drug delivery systems can be prepared in different ways; Drug substance in an adhesive type of delivery system, a drug substance in a matrix type of delivery system or in a reservoir type of delivery system, or by a combining the different types of preparation techniques. The three types of delivery systems offer different ways in controlling the release of drug from the delivery system into the skin.

The formulation principle of the three different systems are described in the following:

Drug Substance in an Adhesive Type of Delivery System

Backing: The backing is a filmforming polymer e.g. containing ethylcellulose, plastic and/or alufoil or other impermeable material.

Adhesive layer: The adhesive layer may contain an adhesive, pressure sensitive polymer layer, e.g. polyacrylate, ethylcellulose or silicone. An enhancer rike; e.g. lauric acid, dioctylcyclohexane, glycerin, n-dodecanol, Eutanol G, isopropylmyristat, PEG 400, propandiol and/or Tween 80 may be added.

Drug: The drug substance can be dissolved or dispersed in the adhesive layer e.g. by a solvent casting or hot melt process. The drug substance can be incorporated in the adhesive layer as microreservoirs.

Liner: A protective liner, to be removed before use, is attached to the adhesive side.

Drug Substance in a Matrix Type Delivery System

Backing: The backing is a filmforming polymer e.g. containing ethycellulose, plastic and/or alufoil or other impermeable material.

Matrix: The matrix may contain a hydrophilic or lipophilic polymer matrix comprising e.g. polyisobutylene. An enhancer like; laurie acid, dioctylcyclohexane, glycerin, n-dodecanol, Eutanol G, isopropylmeristat, PEG 400, propandiol or Tween 80 may be added.

Drug: The drug substance is dispersed in the matrix layer or it may be incorporated in the matrix by a moulding process or be incorporated in the layer as microreservoirs.

Membrane: A rate controlling membrane e.g. of polymeric material might be used to control the diffusion of the drug into the skin. An adhesive layer might be applied on the membrane. The membrane might be omitted.

Adhesive layer: The adhesive layer may contain an adhesive, pressure sensitive polymer layer, e.g. polyacrylate, ethylcellulose or silicone. The adhesive is applied on the rim of the patch, surrounding the matrix-containing drug.

Liner: A protective liner, to be removed before use, is attached to the adhesive side.

Reservoir Type Delivery System

Backing: The backing is a filmforming polymer e.g. containing ethylcellulose, plastic and/or alufoil or other impermeable material.

Reservoir: The reservoir may contain e.g. an unleachable, viscous liquid medium (e.g. silicone fluid) or a releasable solvent (e.g. alkyl alcohol or glycerol) An enhancer like; lauric acid, dioctylcyclohexane, glycerin, n-dodecanol, Eutanol G, isopropylmeristat, PEG 400, propandiol or Tween 80 may be added.

Membrane: A rate controlling membrane e.g. of polymeric material is used to control the diffusion of the drug into the skin. The membrane can be either a microporous or a nonporous membrane e.g. ethylene-vinyl acetate copolymer, with a specific drug permeability. On the external surface of the polymeric membrane a thin layer of drug compatible, pressure-sensitive adhesive copolymer, e.g. silicone adhesive may be applied to ensure the contact to the skin.

Adhesive layer: The adhesive layer may comprise an adhesive, pressure sensitive polymer layer, e.g. polyacrylate, ethylcellulose or silicone. The adhesive is applied on the rim of the patch, surrounding the reservoir-containing drug.

Drug: The drug substance can be dissolved or dispersed in the reservoir layer.

Liner: A protective liner, to be removed before use, is attached to the adhesive side.

### Example 27.1

The following example illustrates a composition of a drug substance (desglymidodrine) in an adhesive transdermal delivery system. A direct contact between the hydrogel and the skin is expected to be essential, to ensure a sufficient flux through stratum corneum.

Backing: The backing is a filmforming polymer e.g. constaining ethylcellulose, plastic and/or alufoil or other impermeable material.

Adhesive layer: The adhesive layer consists of an adhesive, pressure sensitive polyacrylate. The size of the adhesive layer is about 16-20 cm₂, forming a rim around the active layer. The layer contains 0.3-1.0% of desglymidodrine base, to prevent a flux of desglymidodrine base from the active layer to the adhesive layer.

Active layer: The drug substance, desglymidodrine, is dispersed in a hydrogel comprising polyvinylpyrrolidone or partly hydrolised polyvinylic alcohol with a resulting concentration of 15-25% w/w desglymidodrine base. The size of the layer is about 10 cm². A stabiliser like β-cyclodextrin might be added.

Liner: A protective liner, to be removed before use, is attached to the adhesive side.

After removal of the liner the drug delivery system is placed on the skin and the desglymidodrine is delivered for systemic circulation through the skin.

### Example 28

### Pilot Bioavailability Study of Two Prototypes of Midodrine Controlled Release Formulations Compared to Standard Formulation (Tablet) in Healthy Volunteers

Introduction

Systolic blood pressure is transiently and minimally decreased in normal individuals when rising to upright position. Normal physiologic feedback mechanisms work through neurally mediated pathways to maintain the standing blood pressure and thus support adequate cerebral perfusion. These compensatory mechanisms that regulate blood pressure when standing are deficient in patients with orthostatic hypotension, a condition that may lead to inadequate cerebral perfusion with accompanying symptoms of syncope, dizinessalight-headedness and blurred vision, among others.

Midodrine is a prodrug labeled for treatment of orthostatic hypotension. After absorption it is readily metabolized to desglymidodrine that acts as an agonist at the peripheral α-1 receptors in the smooth muscles of arteries and veins, but has no direct central nervous or cardiac effects. Its main effect is to increase the vascular tone thus increasing the total peripheral resistance and rising blood pressure. The presser effect of midodrine is manifest within 20 to 90 minutes after oral administration of a single dose. This pressor effect usually persists for 3 to 6 hours. Doses used in clinical practise (10 mg t.i.d.) significantly increase standing blood pressure, thus alleviating symptoms of orthostatic hypotension.

Controlled Release Formulation

The rationale of the development of a controlled release formulation is to reduce the number of dosings during the day and to avoid major changes in plasma concentration of desglymidodrine. This will increase compliance and reduces changes in severity of symptoms of orthostatic hypotension and thus possibly increase quality of life.

Two prototypes have been developed according to the present invention. One prototype “Micap” is a multiple unit formulation (see Example 12 of PCT application No. PCT/DK01/00214), each unit releasing its amount of midodrine dependent on the acidity of the environment. As the acidity is different in different parts of the gut the result is a continuous release during the passage. The other prototype “Mitab” is composed of three layers releasing midodrine differently creating a time-dependent release (see Example 2 of PCT application No. PCT/DK01/00214). The strength of the controlled release formulations has been chosen to 5 mg to allow for individual titration of the total daily dose. The study is a pilot trial of the bioavailability of the two prototypes compared to a standard tablet.

Objective

To determine the bioavailability of two novel prototypes of controlled release formulations of midodrine hydrochloride compared to standard tablets.

Trial Design

Open-labeled randomized 3 way cross-over trial. All subjects were administered 10 mg midodrine hydrochloride either as a standard tablet or as one of two novel controlled release formulations at three occasions distributed 3 days apart.

Trial Population

Eight healty volunteers, both genders, 18-55 years of age, normal weight, informed consent, not pregnant or lactating, not trying to become pregnant, no liver, renal or gastrointestinal disease that may influence pharmacokinetics or the health of the volunteers, no history of alcohol and drug abuse, non-smokers.

Assessments

AUCₜ (area under the plasma concentration curve to time t), Cₘₐₓ(peak (or shoulder or plateau) plasma concentration), tₘₐₓ (time to peak (or shoulder or plateau) plasma concentration), MRT (mean residence time), t_{>75% Cmax} (W₇₆—duration of plasma concentration above 75% of Cₘₐₓ) HVD (W₅₀—half value duration) and time to a possible second peak (or shoulder or plateau) were calculated for midodrine and its biologically active metabolite, desglymidodrine. AUC_{infinity} (area under the plasma concentration curve extrapolated to infinity) and t_{½} (plasma concentration half life) are calculated, whenever relevant.

Whenever, the concentration went under the detection limit, the values were set to ½×detection limit, i.e. for midodrine ½×1 ng/ml and for desglymidodrine ½×0.5 ng/ml. Because of such a contribution to the AUC, AUC₂₄ was often larger than AUC_{infinity}.

Trial Products

Midodrine tablets 5 mg, Gutron from Nycomed, Denmark, midodrine Controlled release formulation (pH dependent release) prepared as described in Example 12 of PCT application No. PCT/DK01/00214, 5 mg, and midodrine controlled release formulation (time dependent release) prepared as described in Example 2 of PCT application No. PCT/DK01/00214, 5 mg.

Food and Liquid

The subjects were fasting from 8 hours before dosing until 3 hours post dosing. Water was allowed until 1 hour before dosing. No alcoholic beverages or beverages containing caffeine (coffee, tea or cola) are allowed from 8 hours before dosing until last blood sample has been drawn (24 hours).

Study drug was administered to the subjects with 150 ml of water. Additional 150 ml of water was administered to the subjects 1 and 2 hours after dosing.

Meals were standardized throughout all 3 study visits and served according to the following schedule:

4 hours after dosing: lunch

7 hours after dosing: snack

10 hours after dosing: dinner

14 hours after dosing: snack

Study Drug

Two tablets or capsules of study drug (midodrine tablets, Mitab or Micap) (total dose 10 mg) were administered between 7.30 and 8.30 am. Administration of study drug is followed by at least three days washout.

Blood Samples

Seven ml of venous blood were withdrawn immediately before dosing, and at 15 and 30 minutes, 1, 1.5, 2, 3, 5, 6, 7, 8, 9, 10, 11, 12, 14, 16, 20 and 24 hours after dosing. The blood samples were placed on ice immediately after drawing and centrifuged and frozen within 20 minutes. Analysis for midodrine and desglymidodrine was performed by HPLC with fluorescence detection. The analyses were performed by Quintiles AB, Uppsala, Sweden.

Results

The mean plasma concentration curves for midodrine and desglymidodrine, respectively, are shown in FIGS. 3 and 4. The measured and calculated parameters for each composition (n=7 for Micap and Mitab, n=8 for standard tablets) are given in the following.

AUC and MRT have been calculated using the trapezoidal rule and the AUMC method (Yamaoka K., Nakagawa T., Uno T.: Statistical moments in pharmacokinetics, J. Pharmacokin. Biopharm. 1978:6:547-58).

For extrapolation to infinity (the tail) the following formulas have been used:

Cₚ/kₑ (for AUC) and nCₚ/(kₑ²) (for AUMC)

Where Cₚ=the last measured plasma concentration

kₑ=the elimination rate constant

N=the time for last data point with measurable concentrations.

Midodrine ng/ml, mean values (standard deviation):

| | Micap | Mitab | Standard tablets |
|---|---|---|---|
| AUC₂₄ | 32.8 (6.7) | 32.7 (12.1) | 51.7 (13.5) |
| Cₘₐₓ | 10.0 (2.6) | 12.8 (6.5) | 41.4 (12.6) |
| tₘₐₓ | 0.7 (0.4) | 0.9 (0.5) | 0.5 (0.2) |
| MRT | 3.0 (0.4) | 2.3 (0.3) | 1.0 (0.2) |
| HVD | 2.6 (1.1) | 1.5 (0.8) | 0.9 (0.3) |
| t_{>75% Cmax} | 1.0 (1.0) | 0.8 (0.6) | 0.5 (0.1) |
| AUC_{infinity} | 24.1 (6.7) | 21.8 (13.1) | 41.3 (14.0) |

Desglymidodrine ng/ml, mean values (standard deviation):

| | Micap | Mitab | Standard tablets |
|---|---|---|---|
| AUC₂₄ | 106.0 (29.1) | 92.7 (36.4) | 114.4 (31.9) |
| Cₘₐₓ | 11.4 (3.2) | 8.7 (5.0) | 21.7 (5.1) |
| tₘₐₓ | 5.0 (0.0) | 2.9 (1.1) | 1.4 (0.4) |
| MRT | 9.5 (1.0) | 11.8 (4.1) | 4.7 (0.5) |
| HVD | 7.7 (0.4) | 9.9 (4.1) | 4.1 (0.4) |
| t_{>75% Cmax} | 3.4 (0.3) | 4.4 (0.5) | 2.1 (0.4) |
| AUC_{infinity} | 111.5 (33.9) | 104.1 (36.9) | 112.9 (32.5) |

Sum of midodrine and desglymidodrine nmol/l, mean values (standard deviation):

| | Micap | Mitab | Standard tablets |
|---|---|---|---|
| AUC₂₄ | 566.1 (145.6) | 509.1 (195.2) | 667.3 (176.4) |
| Cₘₐₓ | 60.5 (14.5) | 66.0 (29.7) | 195.1 (51.9) |
| tₘₐₓ | 3.0 (1.7) | 1.3 (0.9) | 0.6 (0.2) |
| MRT | 9.7 (1.0) | 12.4 (4.5) | 4.6 (0.6) |
| HVD | 7.7 (0.8) | 5.0 (2.5) | 1.9 (0.8) |
| t_{>75% Cmax} | 4.4 (1.2) | 1.8 (1.3) | 0.9 (0.4) |
| AUC_{infinity} | 608.7 (172.8) | 588.2 (190.1) | 661.1 (183.3) |

Midodrine nmol/l, mean values (standard deviation)

| | Micap | Mitab | Standard tablets |
|---|---|---|---|
| AUC₂₄ | 112.7 (23.2) | 112.5 (41.6) | 178.0 (46.4) |
| Cₘₐₓ | 34.3 (9.1) | 43.9 (22.4) | 142.3 (43.5) |
| tₘₐₓ | 0.7 (0.4) | 0.9 (0.5) | 0.5 (0.2) |
| MRT | 3.0 (0.4) | 2.3 (0.3) | 1.0 (0.2) |
| HVD | 2.6 (1.1) | 1.5 (0.8) | 0.9 (0.3) |
| t_{>75% Cmax} | 1.0 (1.0) | 0.8 (0.6) | 0.5 (0.1) |
| AUC_{infinity} | 83.0 (23.1) | 74.9 (45.0) | 142.1 (48.2) |

Desglymidodrine nmol/l, mean values (standard deviation):

| | Micap | Mitab | Standard tablets |
|---|---|---|---|
| AUC₂₄ | 453.4 (124.7) | 396.6 (156.0) | 489.4 (136.5) |
| Cₘₐₓ | 48.6 (13.5) | 37.4 (21.5) | 92.9 (22.0) |
| tₘₐₓ | 5.0 (0.0) | 2.9 (1.1) | 1.4 (0.4) |
| MRT | 9.5 (1.0) | 11.8 (4.1) | 4.7 (0.5) |
| HVD | 7.7 (0.4) | 9.9 (4.1) | 4.1 (0.4) |
| t_{>75% Cmax} | 3.4 (0.3) | 4.4 (0.5) | 2.1 (0.4) |
| AUC_{infinity} | 477.2 (144.9) | 445.4 (157.7) | 483.3 (138.9) |

Furthermore, the time interval in which the concentration of midodrine, desglymidodrine or the sum of midodrine and desglymidodrine is at a constant value ±40% has been determined. The time interval is found by looking at all possible time intervals (using the time points from the blood sampling) of all possible lengths. For each time interval the mean is calculated and it is checked whether all plasma concentration points in that time interval is lying within ±40% of the mean value. The time interval in question is the longest time interval for which all concentration points in the interval lie within the mean of the time interval ±40%. In order to get a relevant interval the constant value minus 40% has to be higher than the detection limit. The interval is calculated for each patient and the mean value of the length of time interval is given.

For example, for patient 1 the MICAP capsules gave a mean plasma concentration of desglymidodrine at 8.5 ng/ml in the time interval from 2 hours to 9 hours (i.e. a 7 hours interval). In this period the maximum plasma concentration of desglymidodrine was measured as 11.8 ng/ml and the minimum plasma concentration as 5.5 ng/ml. Since 8.5 ng/ml+40% is 11.9 ng/ml and 8.5 ng/ml−40% is 5.1 ng/ml all measured plasma concentration points in that particular interval lie within the mean value ±40%. Since this was the longest time interval where all concentration points lie within the mean ±40% the resulting time interval for the MICAP capsules for patient 1 was 7 hours.

The following results were obtained:

Time interval (hours) where the concentration of midodrine lies at a constant value ±40%:

| | |
|---|---|
| Micap (n = 7) | 1.7 |
| Mitab (n = 7) | 1.4 |
| Standard tablets (n = 8) | 0.63 |

Time interval (hours) where the concentration of desglymidodrine lies at a constant value ±40%:

| | |
|---|---|
| Micap (n = 7) | 6.3 |
| Mitab (n = 7) | 11.5 |
| Standard tablets (n = 8) | 3.7 |

Time interval (hours) where the sum of the concentration of midodrine and desglymidodrine lies at a constant value +40%:

| | |
|---|---|
| Micap (n = 7) | 7.5 |
| Mitab (n = 7) | 11.9 |
| Standard tablets (n = 8) | 3.5 |

The aim of the pilot study was to test the bioavailability of the two novel compositions and a standard Gutron tablet and to estimate whether the compositions are bioequivalent. Furthermore, the controlled release properties of the novel compositions as compared to the standard composition (Gutron tablet) can be depicted from the data generated.

The values of Cₘₐₓ and AUC₀₋₂₄/AUC_{infinity} of the standard tablet are greater than the same values of each of the two prototypes considering the plasma values of midodrine, desglymidodrine and the sum of the two. It is expected that Cₘₐₓ is lower in controlled release compositions than in plain release compositions as this reflects a lesser degree of fluctuation of plasma values. This is one of the purposes of a controlled release composition. It is further supported by the prolongation of the time interval in which the plasma values of midodrine, desglymidodrine and the sum of the concentrations of the two lie at a constant value.

The sum of the plasma concentrations of midodrine and the active metabolite desglymidodrine reflects the total amount of drug absorbed into the blood stream. The values of Tₘₐₓ, W₅₀ and T_{>75% cmax} (W_{75%}) and MRT for this sum concentration are more than 2 times greater of the novel controlled release compositions than the values of the standard tablet. The prolongation of the above mentioned values means that the active drug substance resides in the plasma for a longer time period reducing the numbers of daily dosing needed. Thus, another purpose of a controlled release composition is fulfilled.

Conclusion

Based on the AUC values, the bioavailabilities of the novel controlled release compositions are lesser than the bioavailability of the standard tablet indicating that the content of active drug substance in the controlled release compositions should be increased to establish bioequivalence.

The novel compositions possess controlled release properties as compared to the standard tablet for reasons discussed above.

1. A pharmaceutical composition comprising desglymidodrine or a pharmaceutically acceptable salt thereof together with one or more pharmaceutically acceptable excipients.

2. A composition according to claim 1, wherein desglymidodrine is selected from the group consisting of (±)-α-(aminomethyl)-2,5-dimethoxy-benzenemethanol (±ST 1059), (+)-α-(aminomethyl)-2,5-dimethoxy-benzenemethanol (+ST 1059), (−)-α-(aminomethyl)-2,5-dimethoxy-benzenemethanol (−ST 1059), or mixtures thereof.

3. A composition according to claim 1, wherein desglymidodrine is present in the racemic form (RS), in the enantiomeric form (R), in the enantiomeric form (S) or in mixtures thereof.

4. A composition according to claim 3, wherein the therapeutically active enantiomeric form of desglymidodrine is (−)-α-(aminomethyl)-2,5-dimethoxy-benzenemethanol (−ST 1059) or the (R) form of desglymidodrine ((R) ST 1059).

5. A composition according to any of the preceding claims, wherein at least 90% w/w such as, e.g., at least 95% w/w, at least 97% w/w, at least 98% w/w, at least 99% w/w of desglymidodrine is present in the therapeutically active enantiomeric form.

6. A composition according to any of the preceding claims, wherein desglymidodrine is present in the form of a pharmaceutically acceptable salt such as a salt formed between desglymidodrine and an inorganic acid such as e.g., a hydrochloride, a hydrobromide, a hydrolodide, a nitrate, a nitrite, a H₃PO₃ salt, a H₃PO₄ salt, a H₂SO₃ salt, a sulfate, a H₂SO₅ salt, or a salt formed between desglymidodrine and an organic acid such as organic acids like e.g. H₂CO₃, acetic acid, C₂H₅COOH, C₃H₇COOH, C₄H₉COOH, (COOH)₂, CH₂(COOH)₂, C₂H₅(COOH)₂, C₃H₆(COOH)₂, C₄H₈(COOH)₂, C₅H₁₀(COOH)₂, fumaric acid, maleic acid, lactic acid, citric acid, tartaric acid, ascorbic acid, benzoic acid, salicylic acid and phthalic acid.

7. A composition according to any of the preceding claims for oral, buccal, rectal, nasal, topical, vaginal, ocular or parenteral use.

8. A composition according to any of the preceding claims in the form of a solid, semi-solid or fluid composition.

9. A composition according to claim 8 in solid form, wherein the composition is in the form of tablets such as, e.g. conventional tablets, effervescent tablets, coated tablets, melt tablets or sublingual tablets, pellets, powders, granules, or particulate material

10. A composition according to claim 8 in semi-solid form, wherein the composition is in the form of a chewing gum, an ointment, a cream, a liniment, a paste, a gel or a hydrogel.

11. A composition according to claim 8 in fluid form, wherein the composition is in the form of a solution, an emulsion, a suspension, a dispersion, a liposomal composition, a spray, a mixture, or a syrup.

12. A Composition according to any of claims 1-7 in the form of a delivery device such as, e.g. a lens, a plaster, an implant or a bioadhesive device.

13. A composition according to any of claims 1-11 in unit dosage form such as, e.g., a multiple unit dosage form or a single unit dosage form.

14. A composition according to claim 13, wherein the unit dosage form comprises a daily dose or a part of a daily dose of desglymidodrine.

15. A composition according to any of the preceding claims comprising one or more further active drug substances and/or one or more enhancers.

16. A composition according to claim 15, wherein the further active drug substance is midodrine or a pharmaceutically acceptable salt thereof.

17. A composition according to claim 15, wherein midodrine is present in the form of (±)-2-amino-N-(β-hydroxy-2,5-dimethoxyphenethyl)acetamide, (+)-2-amino-N-(β-hydroxy-2,5-dimethoxyphenethyl)acetamide, (−)-2-amino-N-(β-hydroxy-2,5-dimethoxyphenethyl)acetamide or mixtures thereof.

18. A composition according to claim 15, wherein midodrine is present in the racemic form (RS), in the enantiomeric form (R), in the enantiomeric form (S) or in mixtures thereof

19. A composition according to claim 18, wherein the therapeutically active enantiomeric form of midodrine is (−)-2-amino-N-(β-hydroxy-2,5-dimethoxyphenethyl)acetamide or the (R) form of midodrine.

20. A composition according to claim 18 or 19, wherein at least 90% w/w such as, e.g., at least 95% w/w, at least 97% w/w, at least 98% w/w, at least 99% w/w of midodrine is present in the therapeutically active enantiomeric form.

21. A composition according to claim 9, wherein the composition is in the form of tablets having a disintegration time of at the most about 2.5 min such as, e.g at the most about 30 sec, at the most about 45 sec, at the most about 1 min, at the most about 1.5 min or at the most about 2 min.

22. A composition according to any of the preceding claims, wherein the composition has a shelf-life at room temperature of at least 6 months such as, e.g at least 1 year, at least 1.5 years, at least 2 years, at least 2, 5 years, 3 years, 4 years or 5 years.

23. A composition according to any of the preceding claims, wherein the release kinetics of desglymidodrine from the composition corresponds to that of a plain release tablet.

24. A composition according to any of the preceding claims, wherein the release kinetic of desglymidodrine from the composition corresponding to a zero or a first order release, a mixture of zero and first order release, or any other order of release such as, e.g. 1½, second, third or fourth order release.

25. A composition according to any of the preceding claims, wherein the composition is adapted to release desglymidodrine in such a manner that a relatively fast therapeutic effective concentration of desglymidodrine is obtained after administration of the composition.

26. A composition according to claim 25, wherein the composition is adapted to release desglymidodrine relatively fast in order to obtain an onset of action at the most 15 min after administration such as, e.g. at the most about 1 min, at the most about 2 min, at the most about 3 min, at the most about 4 min, at the most about 5 min, at the most about 7.5 min, at the most about 10 min or at the most about 12.5 min after administration.

27. A composition according to claim 25, wherein the therapeutically effective concentration is obtained within 90 min such as, e.g. within 60 min, within 45 min, within 30 min, within 20 min, within 15 min, within 10 min, within 5 min from administration of the composition.

28. A composition according to claim 25, wherein a relatively fast peak plasma concentration of desglymidodrine is obtained about 1 min-6 hours such as, e.g. about 5 min-6 hours, about 10 min-5 hours, about 15 min-5 hours, about 0.5-6 hours, about 16 hours, about 2-5.5 hours, or about 2.5-5.2 hours after administration.

29. A composition according to any of claims 1-24, wherein the composition is a controlled release composition.

30. A composition according to claim 29, wherein the composition is adapted to provide desglymidodrine in such a manner that a therapeutically effective concentration of desglymidodrine is maintained for at least about 2 hours after administration such as, e.g. at least about 3 hours, at least about 4 hours, at least about 5 hours, at least about 6 hours, at least about 7 hours, at least about 8 hours or at least about 9 hours after administration.

31. A composition according to claim 29, wherein the composition is adapted to release desglymidodrine in such a manner that a therapeutically effective plasma concentrauon of desglymidodrine is maintained for about 4.5-14 hours such as, e.g. about 6-14 hours, about 7-14 hours, about 8-13 hours, about 9-13 hours, about 10-14 hours, about 10-13 hours, or for at least about 4.5 hours, at least about 5 hours, at least about 6 hours, at least about 7 hours, at least about 8 hours, at least about 9 hours, at least about 10 hours, at least about 11 hours, at least about 12 hours, at least about 13 hours or at least about 14 hours.

32. A composition according to claim 31, wherein the plasma concentration of desglymidodrine from the controlled release comnposition is maintained at a relatively constant level for about 4.5-16 hours such as, e.g., 6-14 hours or such as, e.g. for at least about 5 hours, at least about 6 hours, at least about 7 hours, at least about 8 hours, at least about 9 hours, at least about 10 hours, or at least about 11 hours.

33. A composition according to claim 32, wherein the relatively constant level n is ±60%, such as, e.g., n±50%, n±40%, and wherein n is the plasma concentration in ng/ml and monitored in healthy persons.

34. A composition according to any of claims 29-33, wherein the release pattern of desglymidodrine from the controlled release composition—when tested in vitro using Dissolution Method I, II, III, IV, V or VI described herein—is:
1-15% w/w is released from the composition within the first 30 min after start of the test,
10-35% (25%) w/w is released about 30 min after start of the test,
15-40% (35%) w/w is released about 1 hour after start of the test
20-50% (39%) w/w is released about 2 hours after start of the test,
20-55% (47%) w/w is released about 3 hours after start of the test,
25-75% such as, e.g., 25-65% (53%) w/w is released about 4 hours after start of the test,
30-74% (68%) w/w is released about 6 hours after start of the test,
40-95% w/w such as, e.g., 45-85% (80%) w/w is released about 8 hours after start of the test.
65-100% (93%) w/w is released about 10 hours after start of the test,
75-110% (100%) w/w such as, e.g. 90-110% w/w is released about 12 hours after start of the test.

35. A composition according to any of claims 29-34, wherein the composition contains midodrine or a pharmaceutically acceptable salt thereof and wherein the release rate of midodrine from the controlled release composition follows the patterns claimed for desglymidodrine in claim 34.

36. A composition according to claim 29, wherein the composition contains midodrine or a pharmaceutically acceptable salt thereof and wherein the release rate from the controlled release composition of the sum of midodrine and desglymidodrine calculated on a molar basis follows the patterns claimed for midodrine in claim 34.

37. A composition according to claim 29, wherein the controlled release composition comprises at least two parts such as at least a first and a second part, each part contains desglymidodrine and the first part being adapted to release desglymidodrine in a controlled manner during the first 0-14 such as, e.g. 0-11 hours or 0-8 hours after oral intake and the second part being adapted to release desglymidodrine, starting at least 6 hours after oral intake.

38. A composition according to claim 37, wherein at least one of the at least two parts is present in the composition in the form of a multiplicity of individual units such as, e.g. pellets or minitablets.

39. A composition according to claim 37, wherein the two parts of the at least two parts are present in the composition in the form of a multiplicity of individual units such as, e.g. pellets or minitablets, and the two parts are in admixture.

40. A composition according to claim 37, wherein at least one of the at least two parts comprising at least two different types of pellets, the first type of pellets corresponding to a first fraction and the second type of pellets corresponding to a second fraction.

41. A composition according to claim 37, wherein the at least two parts of the composition comprise at least two different types of pellets, the first type of pellets corresponding to the first part and the second type of pellets corresponding to the second part.

42. A composition according to claim 37 in the form of a multiple unit dosage form comprising at least two different types of minitablets, the first type of minitablets corresponding to the first part and the second type of minitablets corresponding to the second part.

43. A composition according to claim 37 further comprising a third part adapted to release desglymidodrine relatively fast from the composition.

44. A composition according to claim 37 further comprising a fourth part adapted to release desglymidodrine from the composition 6-10 hours after administration.

45. A composition according to claim 37 further comprising a fourth part adapted to release desglymidodrine from the composition in the colon after oral intake.

46. A pharmaceutical kit comprising a composition according to any of claims 25-28 and a controlled release composition according to any of claims 29-45.

47. A pharmaceutical kit according to claim 46, comprising
iii) a relatively fast onset pharmaceutical composition according to any of claims 25-28, wherein the composition is adapted to provide desglymidodrine in such a manner that a relatively fast therapeutically effective concentration of desglymidodrine is obtained after administration, and
iv) a controlled release pharmaceutical composition according to any of claims 29-45, wherein the composition is adapted to release desglymidodrine in such a manner that a therapeutically effective plasma concentration of desglymidodrine is maintained for at least about 2 hours, such as, e.g. at least about 3 hours, at least about 4 hours, at least about 5 hours, at least about 6 hours , at least about 7 hours, at least about 8 hours or at least about 9 hours.

48. A pharmaceutical kit according to claims 45 or 46, wherein the relatively fast onset pharmaceutical composition is selected from the group consisting of: oral solid dosage forms, nasal compositions, parenteral compositions, liquid compositions and the like, and the controlled release pharmaceutical composition is selected from the group consisting of oral solid dosage form, transdermal compositions, parenteral composition, vaginal compositions, ocular compositions and the like.

49. A kit according to any of claims 46-48, wherein the relatively fast onset composition or part of the kit results in a peak or shoulder plasma concentration within 90 minutes such as, e.g., within 60 minutes, within 45 minutes, within 30 minutes, or within 20 minutes upon administration of the relatively fast onset composition.

50. A kit according to any of claims 46-49, wherein the relatively fast onset composition is a nasal composition.

51. A kit according to claim 50, wherein the nasal composition comprises polyethyleneglycol and/or glycofurol as a nasal vehicle.

52. A kit according to claim 51, wherein the polyethyleneglycol is PEG 200 and/or PEG 300.

53. A kit according to any claims 46-52, wherein the relatively fast onset composition is in the form of a liposomal composition.

54. A kit according to any of claims 46-49, wherein the relatively fast onset composition is in the form of tablets such as, e.g., melt tablets or sublingual tablets.

55. A kit according to any of claims 46-49, wherein the relatively fast onset composition is a buccal, oral, or rectal composition.

56. A kit according to any of claims 46-55, wherein desglymidodrine in relatively fast onset composition is present in an amount of from 0.2 mg to 10 mg, preferably from 0.5 mg to 7.5 mg such as in an amount of 0.75 mg, 1 mg, 1.25 mg, 1.5 mg, 2 mg, 2.5 mg, 3 mg, 4 mg or 5 mg.

57. A pharmaceutical kit comprising
iii) a relatively fast onset pharmaceutical composition comprising midodrine, wherein the composition is adapted to provide midodrine in such a manner that a relatively fast therapeutically effective concentration of midodrine is obtained after administration, and
iv) a controlled release pharmaceutical composition according to any of claims 29-45, wherein the composition is adapted to release desglymidodrine in such a manner that a therapeutically effective plasma concentration of desglymidodrine is maintained for at least about 2 hours, such as, e.g. at least about 3 hours, at least about 4 hours, at least about 5 hours, at least about 6 hours, at least about 7 hours, at least about 8 hours or at least about 9 hours.

58. A pharmaceutical kit comprising
iii) a relatively fast onset pharnaceutical composition according to any of claims 25-28, wherein the composition is adapted to provide desglymidodrine in such a manner that a relatively fast therapeutically effective concentration of desglymidodrine is obtained after administration, and
iv) a controlled release pharmaceutical composition comprising midodrine, wherein the composition is adapted to release midodrine in such a manner that a therapeutically effective plasma concentration of midodrine is maintained for at least about 2 hours, such as, e.g. at least about 3 hours, at least about 4 hours, at least about 5 hours, at least about 6 hours, at least about 7 hours, at least about 8 hours or at least about 9 hours.

59. A method for treating a patient suffering from orthostatic hypotension and/or urinary incontinence such as urinary stress incontinence, the method comprising administering an effective amount of desglymidodrine in the form of pharmaceutical composition according to any of claims 1-58 to a patient in need thereof.

60. A method according to claim 59, wherein an administration of the composition takes place at wake-up time.

61. A method according to claim 59, wherein an administration of the composition takes place in the morning.

62. A method according to claim 59, wherein an administration of the composition takes place at in the middle of the day and in the form of 1-2 tablets.

63. A method according to any of claims 59-62, wherein the administration takes place 1-3 times daily.

64. A method according to any of claims 59-62, wherein the administration takes place 1 or 2 times daily.

65. A method according to any of claims 59-62, wherein the administration takes place once daily.

66. A method according to any of claims 59-62, wherein the administration of the relatively fast onset composition takes place 1-6 times daily.

67. A method for treating a patient suffering from septic shock, the method comprising administering an effective amount of desglymidodrine in the form of pharmaceutical composition according to any of claims 1-58 to a patient in need thereof.

68. A method according to claim 67, wherein the composition is adapted for parenteral administration.

69. A method according to claims 67 and 68 further comprising a supplemental administration of a composition according to any of claims 1-59.

70. Use of desglymidodrine or a pharmaceutically acceptable salt thereof for the manufacture of a pharmaceutical composition for the treatment of septic shock.

71. A method for treating a patient suffering from a condition responsive to α₁ receptor stimulation, the method comprising administering an effective amount of desglymidodrine in the form of pharmaceutical composition according to any of claims 1-58 to a patient in need thereof.

72. Use of desglymidodrine or a pharmaceutically acceptable salt thereof for the manufacture of a pharmaceutical composition for the treatment of a condition responsive to α₁ receptor stimulation.

73. A method for treating a patient suffering from syncope, the method comprising administering an effective amount of desglymidodrine in the form of pharmaceutical composition according to any of claims 1-58 to a patient in need thereof.

74. Use of desglymidodrine or a pharmaceutically acceptable salt thereof for the manufacture of a pharmaceutical composition for the treatment of syncope.

75. Use of desglymidodrine or a pharmaceutically acceptable salt thereof for the manufacture of a pharmaceutical composition for the treatment of urinary incontinence such as urinary stress incontinence
